(19) United States
(12) Patent Application Publication
(10) Pub. No.: US 2026/0184714 A1
(43) Pub. Date: Jul. 2, 2026

(54) SMALL MOLECULE MODULATORS OF PAR2 AND USES THEREOF

(71) Applicant: PARMEDICS, INC., Temecula, CA (US)

(72) Inventors: Josef VAGNER, Tuscon, AZ (US); Kathryn DEFEA, Riverside, CA (US); Scott A. BOITANO, Tuscon, AZ (US)

(21) Appl. No.: 19/128,120

(22) PCT Filed: Nov. 7, 2023
(86) PCT No.: PCT/US2023/078972

§ 371 Date: May 7, 2025

Related U.S. Application Data
(60) Provisional application No. 63/382,850, filed on Nov. 8, 2022.

(51) Int. Cl. C07D 487/04; A61K 31/4985

(52) U.S. Cl. CPC C07D 487/04; A61K 31/4985

(57) ABSTRACT

(I) or a stereoisomer or salt (e.g., pharmaceutically acceptable salt) thereof, wherein R^{1a}, R^{1b}, R², R^{3a}, R^{3b}, R^{4a}, R^{4b}, R^{5a}, Z, X, and m are as defined herein. Use of the compounds as a component of a pharmaceutical compositions and methods for their use are also provided.

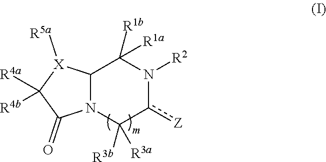

### BACKGROUND

### Technical Field

This invention is in the field of medicinal pharmacology. In particular, the invention relates to a new class of small-molecules having an imidazopyrazine-dione (or related) structure which function as modulators (activators, inhibitors) of protease activated receptor type 2 (PAR₂), and their use as therapeutics for the treatment of conditions involving PAR₂ activity (e.g., asthma, chronic pain, migraine, osteoarthritis, cancer, inflammation disorders and/or vascular disorders).

### Description of the Related Art

Chronic pain is a neurological disorder that impacts the lives of millions of Americans. Chronic pain can be associated with specific diseases, including cancer, or it can be idiopathic with no clear underlying cause. Current treatments for chronic pain are limited by abuse potential and intolerable side effects. Endogenous proteases contribute to acute and chronic pain through the direct activation of PAR₂. PAR₂ is a G-protein coupled receptor (GPCR), known to play an important role in chemical, inflammatory and cancer-induced pain and plays a role in the transition to a chronic pain state (e.g., Bao et al., Mol Pain. 2014 May 5; 10:28; Tian et al., Transl Neurosci. 2015 Mar. 18; 6(1):111-116; Soeda et al., Mol Pain. 2021 January-December; 17:17448069211002009; Tillu et al., Pain. 2015 May; 156(5):859-867). PAR₂ expressed in peripheral nociceptors enhances pain transmission through activation of Transient Receptor Potential (TRP) channels and production of inflammatory mediators, such as neurotransmitters and prostaglandins (e.g., Zhao et al., J Biol Chem. 2014 Sep. 26; 289(39); Hassler et al. JCI Insight. 2020 Jun. 4; 5(11); Grabauskas, et al., Gastroenterology. 2020 June; 158(8):2195-2207). Blocking peripheral PAR₂ activation is a promising approach to limiting chronic non-cancerous and cancer pain, without the abuse risks associated with opioids and with greater efficacy than non-steroidal anti-inflammatory drugs (NSAIDs).

Asthma is a growing and potentially debilitating disease in the industrialized world. Available treatments for asthma have remained constant and novel approaches to therapies are needed. Cellular and animal studies have uncovered prominent roles for airway epithelial PAR₂ in detrimental inflammatory cytokine release and protective ecaisonoid release in response to allergic asthma. These apparently opposing responses can be targeted with novel compounds that individually or collectively modulate the multiple signaling pathways associated with allergen-induced PAR₂ activation.

Osteoarthritis (OA) is the most common chronic pain disorder mostly affecting people later in life. Animal models and clinical data implicate PAR₂ signaling in cells within the damaged joint, and in nociceptors that innervate the joint, contributes to both the underlying inflammation and pain associated with OA. Proteases released from immune cells in OA joints likely activate PAR₂ on these cells making PAR₂ antagonists a viable treatment option for OA.

Migraine pain is a major clinical problem. Almost 15 percent of the global population is affected by migraines during their lifetimes (e.g., Vos, T., et al., Lancet, 2012. 380(9859): p. 2163-96), and there are over 36 million migraine sufferers in the US alone. Even with this significant number of patients, treatments for migraine pain remain little more effective than over-the-counter analgesics. Part of the problem is that migraine etiology is complex and not well understood. Unlike common headaches, migraines have a specific presentation in which a prodrome, aura, and postdrome may occur with the migraine pain lasting between 4 and 72 hours. Hypersensitivity to light and sound, cutaneous allodynia, nausea, and other sensory-motor irregularities are also common symptoms of migraines. It is widely accepted that the trigeminal sensory system, including durally-projecting trigeminal ganglion (TG) nociceptors, is responsible for the pain associated with migraines (e.g., Bernstein, C. and R. Burstein, Journal of clinical neurology, 2012. 8(2): p. 89-99; Levy, D., Headache, 2010. 50(5): p. 909-16). However, it is not understood how the nociceptive afferents from the trigeminal system are activated/sensitized during a migraine attack or where any insults may occur that trigger a migraine attack. It is considered likely that deep cephalic tissues such as the meninges, or possibly the calvarial periosteum, are the tissues involved in nociception during a migraine attack and both have been studied as such in animal models of migraine.

Previous work in the migraine field has shown that degranulation of mast cells in the meninges can release serine proteases which in turn activate PARs and that this response is able to activate dural afferents projecting in the trigeminal nerve (e.g., Zhang, X. C. and D. Levy, Cephalalgia, 2008. 28(3): p. 276-84). Zhang and Levy used single-unit recording electrophysiology to monitor neurons in the trigeminal ganglia of anesthetized rats and applied SLIGRL, a non-specific peptide activator of PAR₂, to the dura of these animals. SLIGRL exposure resulted in activation and sensitization of TG neurons. Injection of the potent and specific PAR₂ agonist, 2aminothiazol-LIGRL or mast cell degranulating agents directly into the dura results in migraine like pain that is absent in PAR₂^{-/-} and inhibited by the PAR₂ antagonist C391 (Hassler S N, et al., Cephalalgia 2019, 39(1):p111-122). This work potentially reveals an important neuro-immune relationship that can explain a wide variety of migraine etiologies since mast cell degranulation can result from cortical spreading depression (CSD), nitric oxide (NO) donors, calcitonin gene-related peptide (CGRP), and heightened stress, all of which are associated with migraine.

Atopic dermatitis (AD) is a common inflammatory skin disease resulting in chronic uncontrollable itch. PAR₂ is activated in keratinocytes by proteases released from pathogens and endogenous proteases such as kallikreins and tryptase, which can lead to activation of temperature sensitive TRP channels, release of inflammatory cytokines and disruption of skin barrier, resulting in itch-like behavior (e.g., Zhao et al., J Invest Dermatol. 2020 August; 140(8):1524-1532; Buhl et al., Front Immunol. 2020 Aug. 12; 11:1740). Currently topical steroids and emollient creams are the most common treatments for AD, but fail to control the itch in many patients. PAR₂ inhibition is a promising potential treatment for AD in patients who are poorly controlled with topical agents.

There is a need for improved methods for treating conditions involving aberrant PAR₂ activity, including chronic pain, asthma, osteoarthritis and migraine. The present disclosure fulfills these needs and offers other related advantages.

### BRIEF SUMMARY

In brief, the present disclosure provides PAR₂ modulator compounds, including stereoisomers or salts (e.g., pharmaceutically acceptable salts) thereof, which can be used alone or in combination with a pharmaceutically acceptable carrier. Methods for use of PAR₂ modulator compounds for treatment of various diseases or conditions, such as asthma, migraine related pain, chronic pain, cancer, and a vascular disorder are also provided.

In one embodiment, compounds having the following Structure (I) are provided:

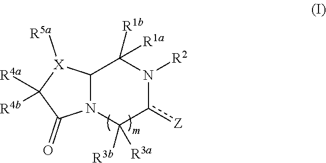

or a stereoisomer or salt thereof, wherein R^{1a}, R^{1b}, R², R^{3a}, R^{3b}, R^{4a}, R^{4b}, R^{5a}, Z, X, and m are as defined herein. Use of the compounds as a component of a pharmaceutical compositions and methods for their use are also provided. Pharmaceutical compositions comprising one or more of the foregoing compounds of Structure (I) and a therapeutic agent are also provided.

In other embodiments, the present disclosure provides a method for administering a therapeutic agent to a patient in need thereof, the method comprising preparing a composition comprising the compound of Structure (I) and a therapeutic agent and delivering the composition to the patient.

These and other aspects of the disclosure will be apparent upon reference to the following detailed description.

### BRIEF DESCRIPTION OF THE DRAWINGS

FIG. 1A shows a graph of normalized cell index versus time for various concentrations of Compound I-1 as an antagonist of PAR2 using the PAR2 antagonist 2-at-LIGRL-NH2 (2AT) as standard;

FIG. 1B shows a graph of normalized cell index versus time for various concentrations of Compound I-1 as an antagonist of PAR2 using trypsin as standard;

FIG. 1C shows a graph of % cells over 150 nM [Ca²⁺] versus various concentrations of Compound I-1 and 2AT as measured by digital imaging microscopy;

FIG. 1D shows a graph of % increase over baseline p-MAPK normalized versus concentration of Compound I-1 in assay for β-arrestin/MAPK signaling;

FIG. 2 shows a graph of facial withdrawal threshold versus time for a series of experiments conducted with Compound I-1; and

FIG. 3 shows a graph of total airway resistance versus amount of administered methacholine for a series of experiments conducted with Compound I-1.

### DETAILED DESCRIPTION

### Compounds

In an aspect, the present disclosure provides PAR₂ modulator compounds, including stereoisomers or salts (e.g., pharmaceutically acceptable salts) thereof, which can be used alone or in combination with a pharmaceutically acceptable carrier. Methods for use of PAR₂ modulator compounds for treatment of various diseases or conditions, such as asthma, migraine related pain, chronic pain, cancer, and a vascular disorder are also provided.

In one embodiment, the compounds have the following Structure (I):

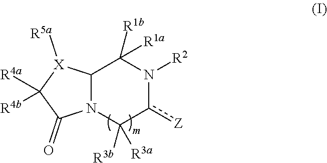

or a stereoisomer or salt thereof, wherein:
R^{1a} and R^{1b} are, each independently, hydrogen, C₁-C₆ alkyl, C₁-C₆ heteroalkyl, C₃-C₈ cycloalkyl, aryl, or arylalkyl;
R² is hydrogen, C₁-C₆ alkyl, C₁-C₆ heteroalkyl, C₃-C₈ heterocyclylalkyl, C₃-C₈ heterocyclylalkylC₁₋₆alkyl, heteroaryl, heteroarylalkyl, or arylalkyl;
Z is either i) oxygen when [symbol] is a double bond or ii) hydrogen, C₁-C₆ alkyl, C₁-C₆ heteroalkyl, C₃-C₈ cycloalkyl, or arylalkyl when [symbol] is a single bond;
R^{3a} and R^{3b} are, each independently, hydrogen, C₁-C₆ alkyl, C₁-C₆ heteroalkyl, C₃-C₈ cycloalkyl, or arylalkyl;
X is N or C(R^{5b});
either
   i) R^{4a} and R^{4b} are, each independently, hydrogen, C₁-C₆ alkyl, C₁-C₆ heteroalkyl, C₃-C₈ cycloalkyl, C₃-C₈ heterocyclylalkyl, arylalkyl, or heteroarylalkyl; and
   R^{5a} and R^{5b} are, each independently, hydrogen, C₁-C₆ alkyl, C₁-C₆ hydroxyalkyl, C₃-C₈ cycloalkyl, C₃-C₈ heterocyclylalkyl, arylalkyl, heteroarylalkyl, —C(═O)R⁶, —C(═O)YR⁶, —OC(═O)R⁶, —OC(═O)YR⁶, —NHC(═O)R⁶, or —NHC(═O)YR⁶, provided that when R^{5a} and R^{5b} are, each independently, —NHC(═O)R⁶, or —NHC(═O)YR⁶, R⁶ is not an arylalkyl; or
   ii) R^{4a} forms a direct bond with X and R^{4b} and R^{5b} together form a 5-9 member fused ring;
R⁶ is C₃-C₈ cycloalkyl, C₃-C₈ heterocyclylalkyl, arylalkyl, heteroarylalkyl, or a fused ring;
Y is O, S, or NR⁷;
R⁷ is hydrogen, hydroxyl, or C₁-C₆ alkyl;
m is 1, or 2; and
wherein each of R^{1a}, R^{1b}, R², R^{3a}, R^{3b}, R^{4a}, R^{4b}, R^{5a}, R^{5b}, R⁶, and R⁷ is optionally substituted with one or more substituent.

In some embodiments, m is 1. In some other embodiments, m is 2.

In some embodiments, X is N. In certain embodiments, the compound having the following Structure (Ia):

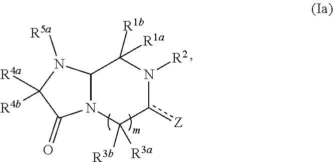

or a stereoisomer or salt thereof.

In some embodiments, X is N, and Z is oxygen. In certain embodiments, the compound having the following Structure (Ib):

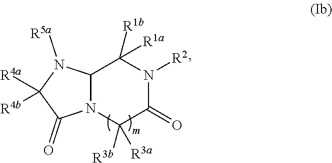

or a stereoisomer or salt thereof.

In some other embodiments, m is 2 and X is N. In certain embodiments, the compound having the following Structure (Ic).

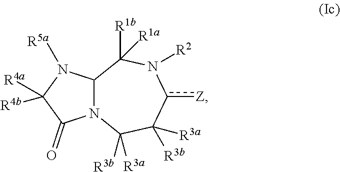

or a stereoisomer or salt thereof.

In some embodiments, m is 2, X is N, and Z is oxygen. In certain embodiments, the compound having the following Structure (Id):

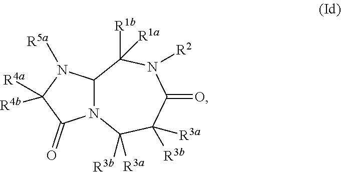

or a stereoisomer or salt thereof.

In some embodiments, X is C(R^{5b}) and the compound having the following Structure (Ij):

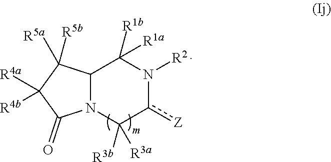

In some embodiments, R^{4a} forms a direct bond with X and R^{4b} and R^{5b} together form a 5-9 member fused ring. In some certain embodiments, wherein the compound having one of the following Structures (Ie)-(Ih):

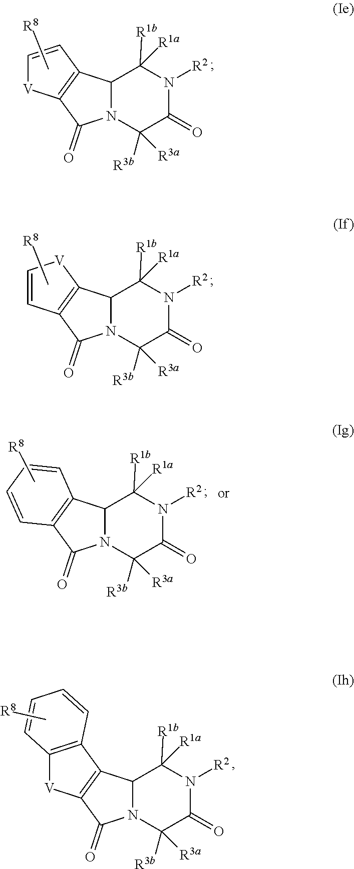

or a stereoisomer or salt thereof, wherein R⁸ is hydrogen, halo, hydroxyl, alkoxy, amino, cyano, C₁-C₆ alkyl, or C₁-C₆ heteroalkyl; and V is a carbon, nitrogen, or oxygen atom.

In some embodiments, R^{1a} or R^{1b} is hydrogen. In some specific embodiments, each of R^{1a} and R^{1b} are hydrogen. In some embodiments, R^{1a} or R^{1b} is C₁-C₆ alkyl. In some other embodiments, R^{1a} or R^{1b} is C₁-C₆ heteroalkyl. In some other embodiments, R^{1a} or R^{1b} is C₃-C₈ cycloalkyl. In some other embodiments, R^{1a} or R^{1b} is aryl. In some other embodiments, R^{1a} or R^{1b} is arylalkyl. In some other embodiments, R^{1a} and R^{1b} have one of the following structures:

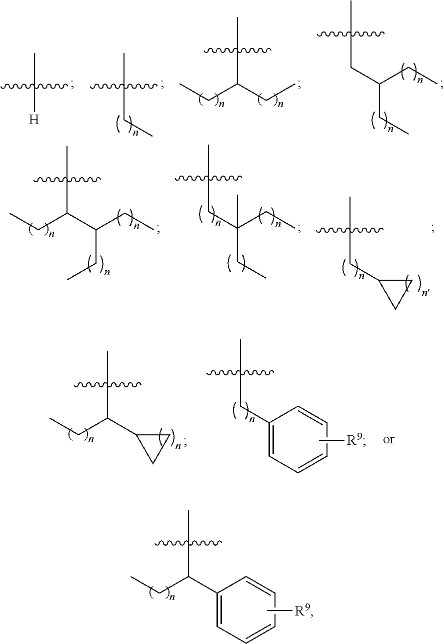

wherein n is, each independently, 0-5 and n′ is 1-4; and R⁹ is hydrogen, halo, hydroxyl, alkoxy, amino, cyano, C₁-C₆ alkyl, or C₁-C₆ heteroalkyl.

In some embodiments, n is 0, 1, 2, 3, 4, or 5. In some embodiments, n is 0. In some other embodiments, n is 1. In some other embodiments, n is 2. In some other embodiments, n is 3. In some other embodiments, n is 4. In some other embodiments, n is 5.

In some embodiments, n′ is 1, 2, 3, or 4. In some embodiments, n′ is 1. In certain embodiments, when n′ is 1 and it is cyclopropane. In some other embodiments, n′ is 2. In certain embodiments, when n′ is 2 and it is cyclobutane. In some other embodiments, n′ is 3. In certain embodiments, when n′ is 3 and it is cyclopentane. In some other embodiments, n′ is 4. In certain embodiments, when n′ is 4 and it is cyclohexane.

In some embodiments, R⁹ is hydrogen. In some other embodiments, R⁹ is halo. In some other embodiments, R⁹ is hydroxyl. In some other embodiments, R⁹ is alkoxy. In some other embodiments, R⁹ is amino. In some other embodiments, R⁹ is cyano. In some other embodiments, R⁹ is C₁-C₆ alkyl. In some other embodiments, R⁹ is C₁-C₆ heteroalkyl.

In some embodiments, R² is hydrogen. In some other embodiments, R² is C₁-C₆ alkyl. In some other embodiments, R² is C₁-C₆ heteroalkyl. In some other embodiments, R² is C₃-C₈ heterocyclylalkyl. In some other embodiments, R² is C₃-C₈ heterocyclylalkylC₁₋₆alkyl. In some other embodiments, R² is heteroaryl. In some other embodiments, R² is heteroarylalkyl. In some other embodiments, R² is arylalkyl. In some embodiments, R² has one of the following structures:

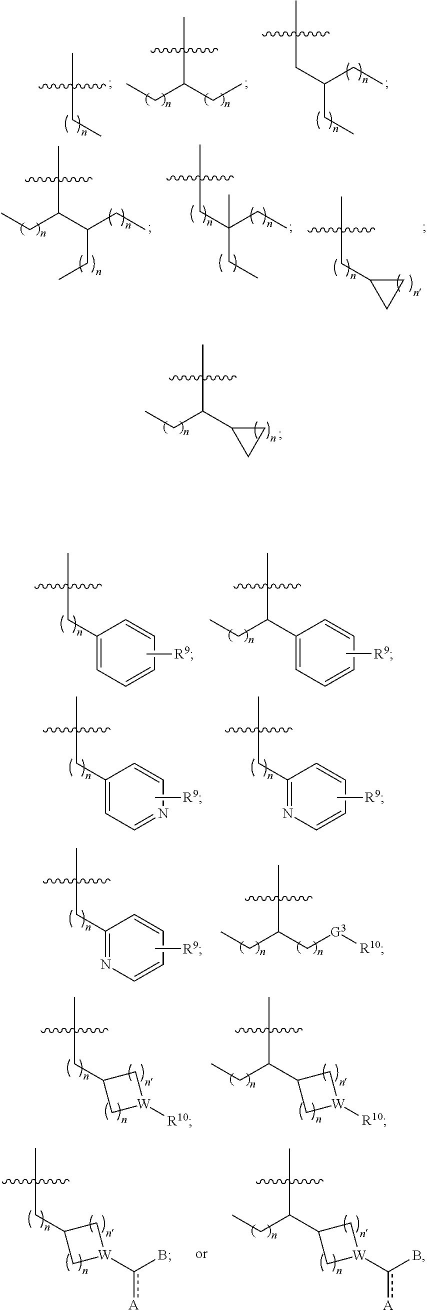

wherein n is, each independently 0-6 and n′ is 1-5; G² is CH₂, N, O or S W is CH or N; A is O, NH, or NHR⁹, wherein i) [symbol] is a double bond when A is O or ii) [symbol] is a single bond when A is NH or NHR⁹; B is OR⁹, NH₂, or NHR⁹; R⁹ is hydrogen, halo, hydroxyl, alkoxy, amino, cyano, C₁-C₆ alkyl, or C₁-C₆ heteroalkyl; and R¹⁰ is either i) absent or ii) hydrogen, alkyl, aryl, pyridyl, acylalkyl, or acylaryl.

In some specific embodiments, R² is

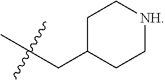 [SMILES: *CC1CCNCC1]

In some embodiments, Z is oxygen when [symbol] is a double bond. In some specific embodiments, when Z is oxygen, the compound has one of the following structures:

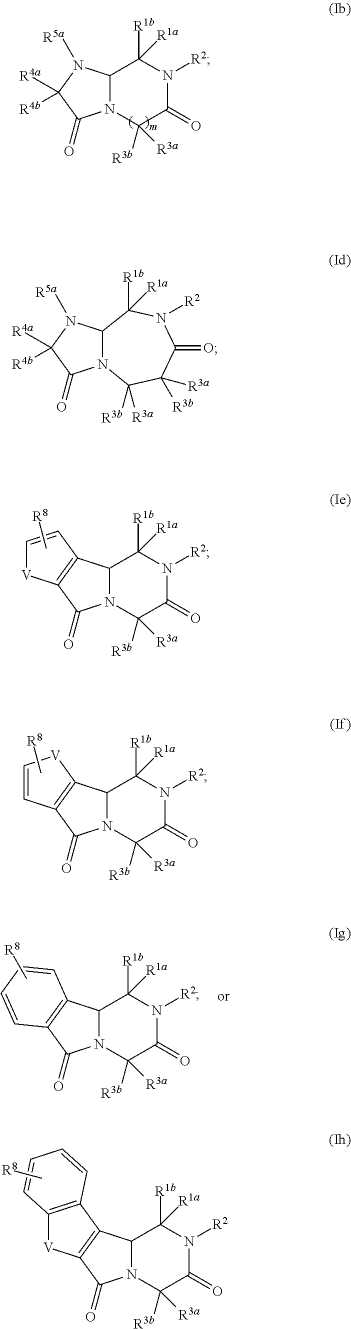

In some other embodiments, Z is hydrogen when [symbol] is a single bond. In some embodiments, Z is C₁-C₆ alkyl when [symbol] is a single bond. In some embodiments, Z is C₁-C₆ heteroalkyl when [symbol] is a single bond. In some embodiments, Z is C₃-C₈ cycloalkyl when [symbol] is a single bond. In some embodiments, Z is arylalkyl when [symbol] is a single bond. In some embodiments, when [symbol] is a single bond, the compound has one of the following structures:

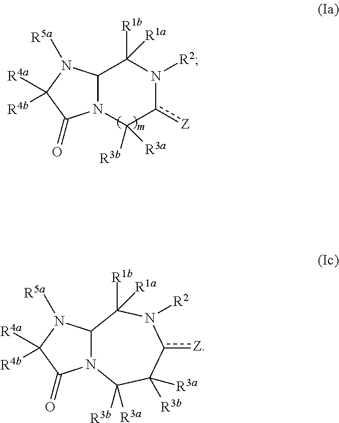

In some embodiments, R^{3a} or R^{3b} is hydrogen. In some other embodiments, R^{3a} or R^{3b} is C₁-C₆ alkyl. In some other embodiments, R^{3a} or R^{3b} is C₁-C₆ heteroalkyl. In some other embodiments, R^{3a} or R^{3b} is C₃-C₈ cycloalkyl. In some other embodiments, R^{3a} or R^{3b} is arylalkyl. In some embodiments, R^{3a} or R^{3b} has one of the following structures:

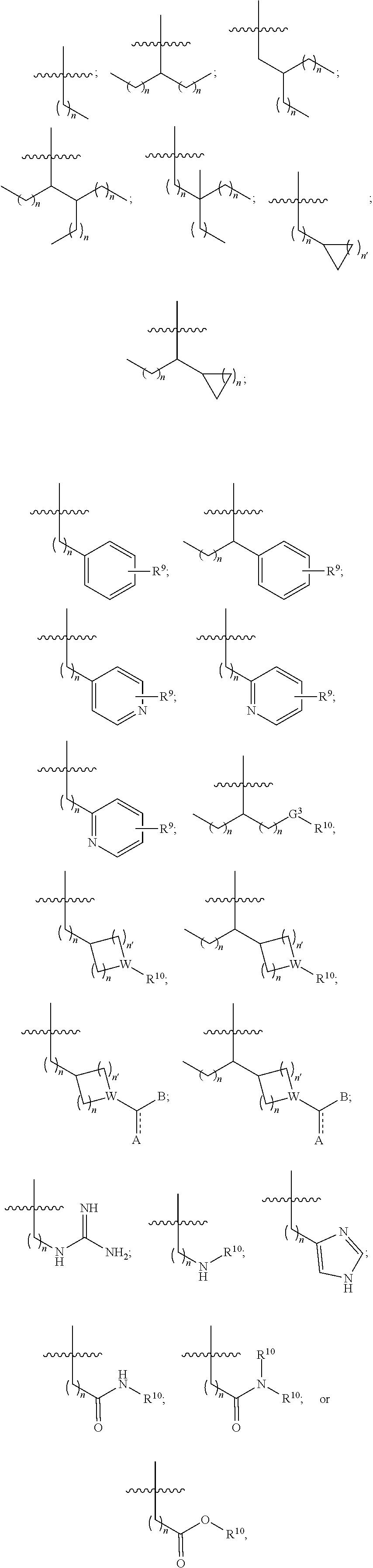

wherein n is, each independently, 0-6 and n′ is 1-5; G³ is, each independently, C(R⁷)₂, O, S, or NR⁷; W is, each independently, CH or N; A is O, NH, or NHR⁹, wherein i) [symbol] is a double bond when A is O or ii) [symbol] is a single bond when A is NH or NHR⁹; B is OR⁹, NH₂, or NHR⁹; R⁹ is hydrogen, halo, hydroxyl, alkoxy, amino, cyano, C₁-C₆ alkyl, or C₁-C₆ heteroalkyl; and R¹⁰ is either i) absent or ii) hydrogen, alkyl, aryl, pyridyl, acylalkyl, or acylaryl.

In some specific embodiments, one of R^{3a} or R^{3b} is hydrogen and the other one of R^{3a} or R^{3b} is

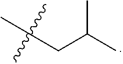 [SMILES: *CC(C)C]

In some other specific embodiments, one of R^{3a} or R^{3b} is hydrogen and the other one of R^{3a} or R^{3b} is

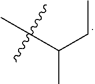 [SMILES: *C(C)CC]

In some embodiments, R^{4a} or R^{4b} is hydrogen. In some other embodiments, R^{4a} or R^{4b} is C₁-C₆ alkyl. In some other embodiments, R^{4a} or R^{4b} is C₁-C₆ heteroalkyl. In some other embodiments, R^{4a} or R^{4b} is C₃-C₈ cycloalkyl. In some other embodiments, R^{4a} or R^{4b} is C₃-C₈ heterocyclylalkyl. In some other embodiments, R^{4a} or R^{4b} is arylalkyl. In some other embodiments, R^{4a} or R^{4b} is heteroarylalkyl. In some embodiments, R^{4a} or R^{4b} has one of the following structures:

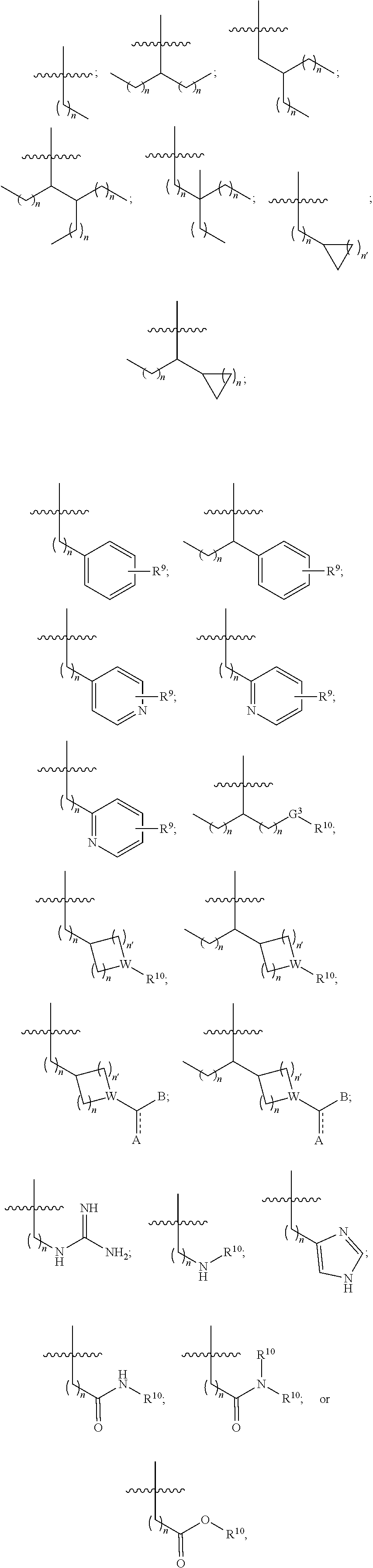

wherein n is, each independently, 0-6 and n′ is 1-5; G⁴ is, each independently, C(R⁷)₂, O, S, or NR⁷; W is, each independently, CH or N; A is O, NH, or NHR⁹, wherein i) [symbol] is a double bond when A is O or ii) [symbol] is a single bond when A is NH or NHR⁹; B is OR⁹, NH₂, or NHR⁹; R⁹ is hydrogen, halo, hydroxyl, alkoxy, amino, cyano, C₁-C₆ alkyl, or C₁-C₆ heteroalkyl; and R¹⁰ is either i) absent or ii) hydrogen, alkyl, aryl, pyridyl, acylalkyl, or acylaryl.

In some specific embodiments, R^{4a} or R^{4b} is hydrogen and the other one of R^{4a} or R^{4b} is

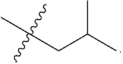 [SMILES: *CC(C)C]

In some embodiments, R^{5a} or R^{5b} is hydrogen. In some other embodiments, R^{5a} or R^{5b} is C₁-C₆ alkyl. In some other embodiments, R^{5a} or R^{5b} is C₁-C₆ hydroxyalkyl. In some other embodiments, R^{5a} or R^{5b} is C₃-C₈ cycloalkyl. In some other embodiments, R^{5a} or R^{5b} is C₃-C₈ heterocyclylalkyl. In some other embodiments, R^{5a} or R^{5b} is arylalkyl. In some other embodiments, R^{5a} or R^{5b} is heteroarylalkyl. In some embodiments, R^{5a} or R^{5b} is —C(═O)R⁶. In some other embodiments, R^{5a} or R^{5b} is —C(═O)YR⁶. In some other embodiments, R^{5a} or R^{5b} is —OC(═O)R⁶. In some other embodiments, R^{5a} or R^{5b} is —OC(═O)YR⁶. In some other embodiments, R^{5a} or R^{5b} is —NHC(═O)R⁶. In some other embodiments, R^{5a} or R^{5b} is —NHC(═O)YR⁶. In some embodiments, R^{5a} has one of the following structures:

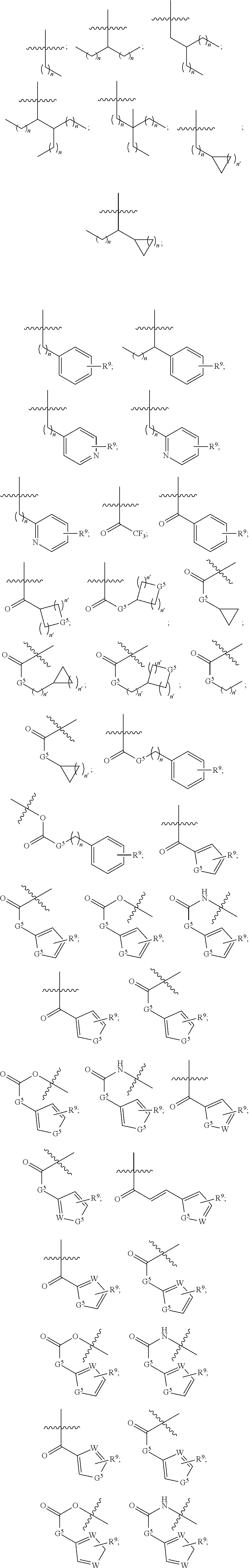

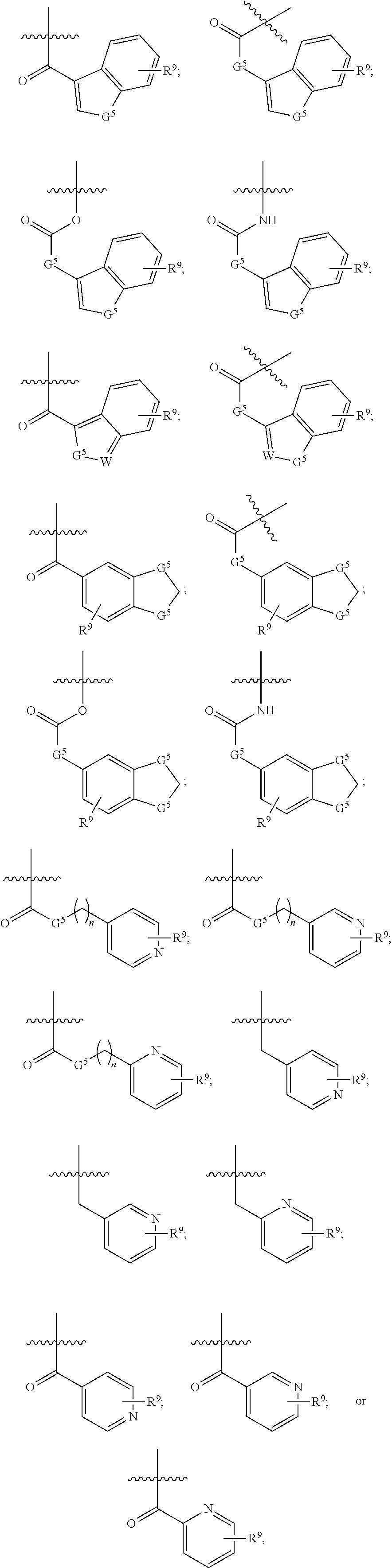

wherein n is 0-6 and n′ is 1-5; G⁵ is, each independently, C(R⁷)₂, O, S, or NR⁷; and W is, each independently, CH or N.

In some embodiments, n is 0. In some other embodiments, n is 1. In some other embodiments, n is 2. In some other embodiments, n is 3. In some other embodiments, n is 4. In some other embodiments, n is 5. In some other embodiments, n is 6.

In some embodiments, n′ is 1. In some other embodiments, n′ is 2. In some other embodiments, n′ is 3. In some other embodiments, n′ is 4. In some other embodiments, n′ is 5.

In some embodiments, W is CH. In some other embodiments, W is C(R⁷)₂. In some other embodiments, W is O. In some other embodiments, W is N. In some other embodiments, W is S. In some other embodiments, W is NR⁷.

In some embodiments, R⁷ is hydrogen. In some other embodiments, R⁷ is hydroxyl. In some other embodiments, R⁷ is C₁-C₆ alkyl.

In some embodiments, R⁹ is hydrogen. In some other embodiments, R⁹ is halo. In some other embodiments, R⁹ is hydroxyl. In some other embodiments, R⁹ is alkoxy. In some other embodiments, R⁹ is amino. In some other embodiments, R⁹ is cyano. In some other embodiments, R⁹ is C₁-C₆ alkyl. In some other embodiments, R⁹ is C₁-C₆ heteroalkyl.

In some specific embodiments, R^{5a} is

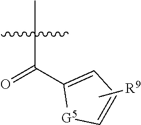

and R^{5b} is hydrogen. In certain embodiments, Z is O and R⁹ is hydrogen. In some embodiments, R^{5b}. has the following structure:

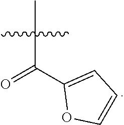 [SMILES: *C(=O)c1ccco1]

In some embodiments, R^{5a} has one of the following structures:

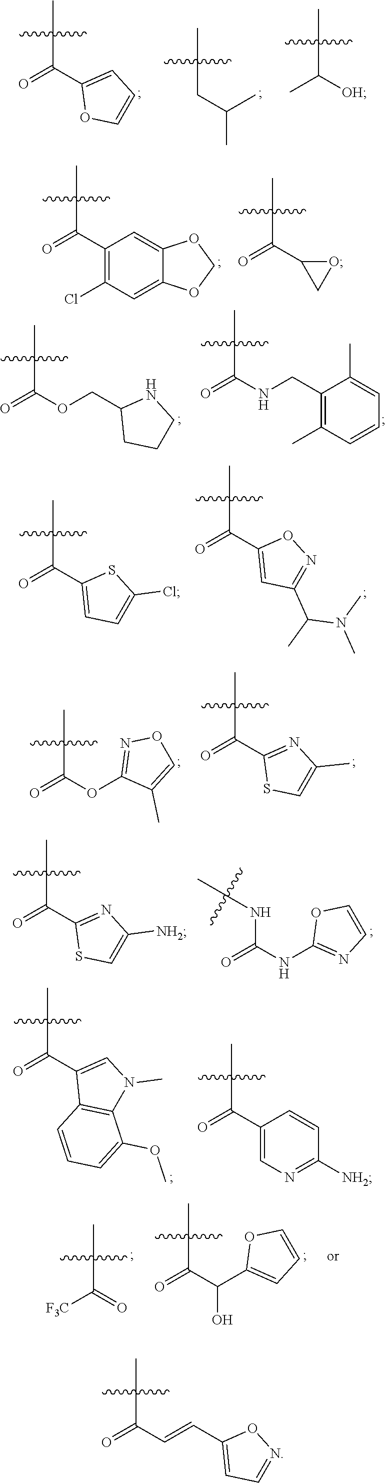

In some embodiments, R⁶ is C₃-C₈ cycloalkyl. In some other embodiments, R⁶ is C₃-C₈ heterocyclylalkyl. In some other embodiments, R⁶ is arylalkyl. In some embodiments, R⁶ is heteroarylalkyl. In some embodiments, R⁶ is a 5-9 member fused ring.

In some embodiments, the compound is a free base form. In certain embodiments, the compound is a pharmaceutically acceptable salt. In certain embodiments, the compound is a tautomer.

In various different embodiments, the compound has one of the structures set forth in Table 1 below (or a stereoisomer or salt thereof).

TABLE 1
Representative compounds of Structures (I) and (Ib)

| No. | Compound | Name |
|---|---|---|
| I-1 | 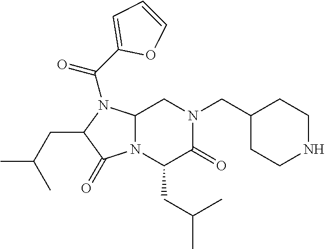 [SMILES: CC(C)CC1C(=O)N2C(CN(CC3CCNCC3)C(=O)[C@@H]2CC(C)C)N1C(=O)c1ccco1] | (5S)-1-(furan-2-carbonyl)- 2,5-diisobutyl-7-(piperidin-4- ylmethyl)tetrahydroimidazo[1,2- a]pyrazine-3,6(2H,5H)- dione |
| I-2 | 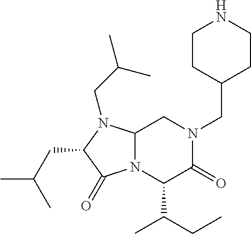 [SMILES: CCC(C)[C@H]1C(=O)N(CC2CCNCC2)CC2N(CC(C)C)[C@@H](CC(C)C)C(=O)N21] | (2S,5S)-5-((R)-sec-butyl)-1,2- diisobutyl-7-(piperidin-4- ylmethyl)tetrahydroimidazo[1,2- a]pyrazine-3,6(2H,5H)- dione |
| I-3 | 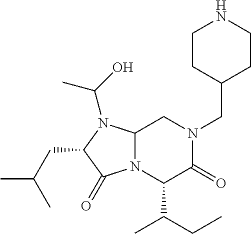 [SMILES: CCC(C)[C@H]1C(=O)N(CC2CCNCC2)CC2N1C(=O)[C@H](CC(C)C)N2C(C)O] | (2S,5S)-5-((R)-sec-butyl)-1- (1-hydroxyethyl)-2-isobutyl- 7-(piperidin-4- ylmethyl)tetrahydroimidazo[1,2- a]pyrazine-3,6(2H,5H)- dione |
| I-4 | 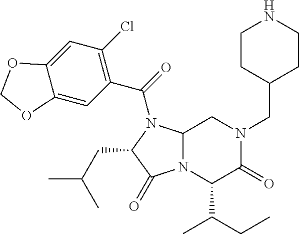 [SMILES: CCC(C)[C@H]1C(=O)N(CC2CCNCC2)CC2N1C(=O)[C@H](CC(C)C)N2C(=O)c1cc2c(cc1Cl)OCO2] | (2S,5S)-5-((R)-sec-butyl)-1- (6- chlorobenzo[d][1,3]dioxole-5- carbonyl)-2-isobutyl-7- (piperidin-4- ylmethyl)tetrahydroimidazo[1,2- a]pyrazine-3,6(2H,5H)- dione |
| I-5 | 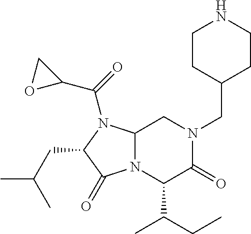 [SMILES: CCC(C)[C@H]1C(=O)N(CC2CCNCC2)CC2N1C(=O)[C@H](CC(C)C)N2C(=O)C1CO1] | (2S,5S)-5-((R)-sec-butyl)-2- isobutyl-1-(oxirane-2- carbonyl)-7-(piperidin-4- ylmethyl)tetrahydroimidazo[1,2- a]pyrazine-3,6(2H,5H)- dione |
| I-6 | 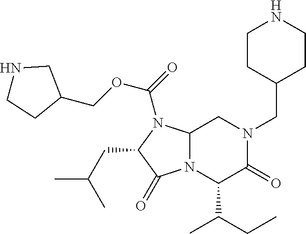 [SMILES: CCC(C)[C@H]1C(=O)N(CC2CCNCC2)CC2N1C(=O)[C@H](CC(C)C)N2C(=O)OCC1CCNC1] | pyrrolidin-3-ylmethyl (2S,5S)-5-((R)-sec-butyl)-2- isobutyl-3,6-dioxo-7- (piperidin-4- ylmethyl)hexahydroimidazo[1,2- a]pyrazine-1(5H)- carboxylate |
| I-7 | 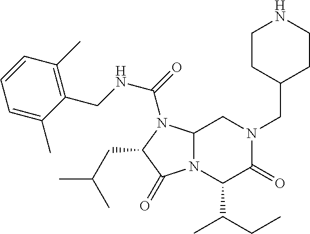 [SMILES: CCC(C)[C@H]1C(=O)N(CC2CCNCC2)CC2N1C(=O)[C@H](CC(C)C)N2C(=O)NCc1c(C)cccc1C] | (2S,5S)-5-((R)-sec-butyl)-N- (2,6-dimethylbenzyl)-2- isobutyl-3,6-dioxo-7- (piperidin-4- ylmethyl)hexahydroimidazo[1,2- a]pyrazine-1(5H)- carboxamide |
| I-8 | 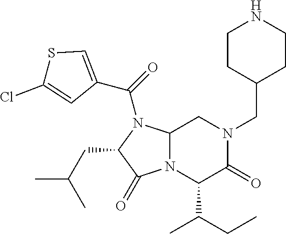 [SMILES: CCC(C)[C@H]1C(=O)N(CC2CCNCC2)CC2N1C(=O)[C@H](CC(C)C)N2C(=O)c1csc(Cl)c1] | (2S,5S)-5-((R)-sec-butyl)-1- (5-chlorothiophene-3- carbonyl)-2-isobutyl-7- (piperidin-4- ylmethyl)tetrahydroimidazo[1,2- a]pyrazine-3,6(2H,5H)- |
| I-9 | 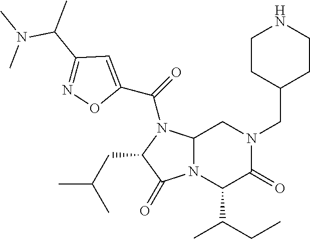 [SMILES: CCC(C)[C@H]1C(=O)N(CC2CCNCC2)CC2N1C(=O)[C@H](CC(C)C)N2C(=O)c1cc(C(C)N(C)C)no1] | (2S,5S)-5-((R)-sec-butyl)-1- (3-(1- (dimethylamino)ethyl)isoxazole- 5-carbonyl)-2-isobutyl-7- (piperidin-4- ylmethyl)tetrahydroimidazo[1,2- a]pyrazine-3,6(2H,5H)- dione |
| I-10 | 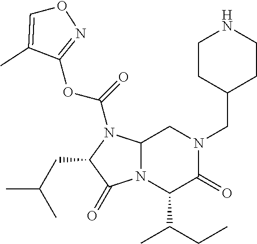 [SMILES: CCC(C)[C@H]1C(=O)N(CC2CCNCC2)CC2N1C(=O)[C@H](CC(C)C)N2C(=O)Oc1nocc1C] | 4-methylisoxazol-3-yl (2S,5S)-5-((R)-sec-butyl)-2- isobutyl-3,6-dioxo-7- (piperidin-4- ylmethyl)hexahydroimidazo[1,2- a]pyrazine-1(5H)- carboxylate |
| I-11 | 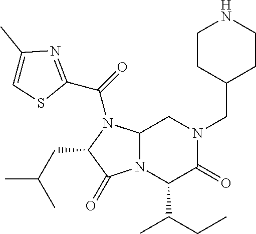 [SMILES: CCC(C)[C@H]1C(=O)N(CC2CCNCC2)CC2N1C(=O)[C@H](CC(C)C)N2C(=O)c1nc(C)cs1] | (2S,5S)-5-((R)-sec-butyl)-2- isobutyl-1-(4-methylthiazole- 2-carbonyl)-7-(piperidin-4- ylmethyl)tetrahydroimidazo[1,2- a]pyrazine-3,6(2H,5H)- dione |
| I-12 | 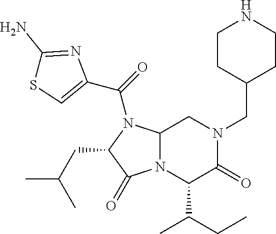 [SMILES: CCC(C)[C@H]1C(=O)N(CC2CCNCC2)CC2N1C(=O)[C@H](CC(C)C)N2C(=O)c1csc(N)n1] | (2S,5S)-1-(2-aminothiazole-4- carbonyl)-5-((R)-sec-butyl)-2- isobutyl-7-(piperidin-4- ylmethyl)tetrahydroimidazo[1,2- a]pyrazine-3,6(2H,5H)- dione |
| I-13 | 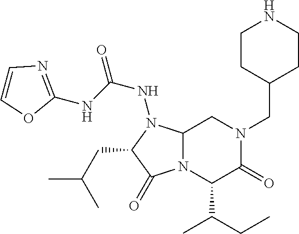 [SMILES: CCC(C)[C@H]1C(=O)N(CC2CCNCC2)CC2N(NC(=O)Nc3ncco3)[C@@H](CC(C)C)C(=O)N21] | 1-((2S,5S)-5-((R)-sec-butyl)- 2-isobutyl-3,6-dioxo-7- (piperidin-4- ylmethyl)hexahydroimidazo[1,2- a]pyrazin-1(5H)-yl)-3- (oxazol-2-yl)urea |
| I-14 | 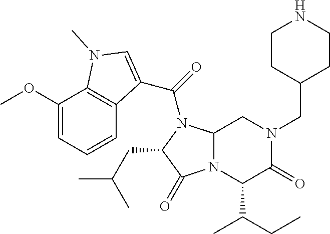 [SMILES: CCC(C)[C@H]1C(=O)N(CC2CCNCC2)CC2N1C(=O)[C@H](CC(C)C)N2C(=O)c1cn(C)c2c(OC)cccc12] | (2S,5S)-5-((R)-sec-butyl)-2- isobutyl-1-(7-methoxy-1- methyl-1H-indole-3- carbonyl)-7-(piperidin-4- ylmethyl)tetrahydroimidazo[1,2- a]pyrazine-3,6(2H,5H)- dione |
| I-15 | 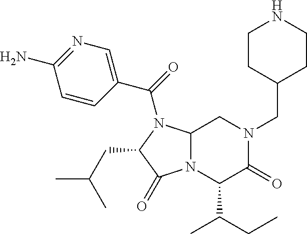 [SMILES: CCC(C)[C@H]1C(=O)N(CC2CCNCC2)CC2N1C(=O)[C@H](CC(C)C)N2C(=O)c1ccc(N)nc1] | (2S,5S)-1-(6- aminonicotinoyl)-5-((R)-sec- butyl)-2-isobutyl-7-(piperidin- 4- ylmethyl)tetrahydroimidazo[1,2- a]pyrazine-3,6(2H,5H)- dione |
| I-16 | 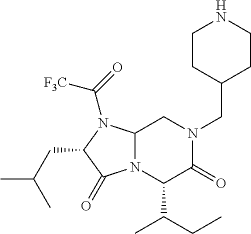 [SMILES: CCC(C)[C@H]1C(=O)N(CC2CCNCC2)CC2N1C(=O)[C@H](CC(C)C)N2C(=O)C(F)(F)F] | (2S,5S)-5-((R)-sec-butyl)-2- isobutyl-7-(piperidin-4- ylmethyl)-1-(2,2,2- trifluoroacetyl)tetrahydroimida- zo[1,2-a]pyrazine- 3,6(2H,5H)-dione |
| I-17 | 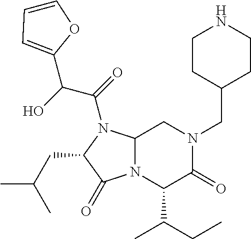 [SMILES: CCC(C)[C@H]1C(=O)N(CC2CCNCC2)CC2N1C(=O)[C@H](CC(C)C)N2C(=O)C(O)c1ccco1] | (2S,5S)-5-((R)-sec-butyl)-1- (2-(furan-2-yl)-2- hydroxyacetyl)-2-isobutyl-7- (piperidin-4- ylmethyl)tetrahydroimidazo[1,2- a]pyrazine-3,6(2H,5H)- dione |
| I-18 | 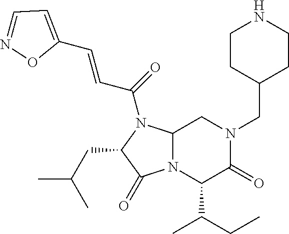 [SMILES: CCC(C)[C@H]1C(=O)N(CC2CCNCC2)CC2N1C(=O)[C@H](CC(C)C)N2C(=O)/C=C/c1ccno1] | (2S,5S)-5-((R)-sec-butyl)-2- isobutyl-1-((E)-3-(isoxazol-5- yl)acryloyl)-7-(piperidin-4- ylmethyl)tetrahydroimidazo[1,2- a]pyrazine-3,6(2H,5H)- dione |

It is understood that any embodiment of the compounds of Structures (I)-(Ih) as set forth above, and any specific substituent and/or variable in the compound of Structures (I)-(Ih) as set forth above may be independently combined with other embodiments and/or substituents and/or variables of compounds of Structures (I)-(Ih) to form embodiments of the disclosure not specifically set forth above. In addition, in the event that a list of substituents and/or variables is listed for any particular R group or variables n or m in a particular embodiment and/or claim, it is understood that each individual substituent and/or variable may be deleted from the particular embodiment and/or claim and that the remaining list of substituents and/or variables will be considered to be within the scope of the disclosure. It is understood that in the present description, combinations of substituents and/or variables of the depicted formulae are permissible only if such contributions result in stable compounds.

In the description, certain specific details are set forth in order to provide a thorough understanding of various embodiments of the disclosure. However, one skilled in the art will understand that embodiments of the disclosure may be practiced without these details. As used herein, the following terms have the meanings ascribed to them unless specified otherwise.

Unless the context requires otherwise, throughout the present specification and claims, the word “comprise” and variations thereof, such as, “comprises” and “comprising” are to be construed in an open and inclusive sense, that is, as “including, but not limited to”.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Hydroxy” or “hydroxyl” refers to an —OH radical.

“Amino” refers to an —NH₂ radical.

“Cyano” refers to a —CN radical.

“Alkyl” refers to a saturated straight or branched hydrocarbon chain radical consisting solely of carbon and hydrogen atoms having from one to six carbon atoms (C₁-C₆ alkyl), which is attached to the rest of the molecule by a single bond. Hydrocarbon chain radicals include, for example, methyl, ethyl, n-propyl, 1 methylethyl (iso-propyl), n-butyl, n-pentyl, 1,1 dimethylethyl (t-butyl), iso-pentyl, n-hexyl, and the like. Unless stated otherwise specifically in the specification, an alkyl group is optionally substituted.

“Cycloalkyl” refers to a saturated cyclic hydrocarbon radical having from three to eight carbon atoms (C₃-C₈ cycloalkyl) attached to the rest of the molecule by a single bond. Saturated cyclic hydrocarbon radicals include, for example, cyclopropyl, cyclobutyl, cyclopentyl or cyclohexyl. Unless otherwise stated specifically in the specification, a cycloalkyl group is optionally substituted.

“Halo” refers to fluoro, chloro, bromo, or iodo. Halo belongs to group 17 of the periodic table.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Haloalkoxy” refers to a radical having the following formula: —Ohaloalkyl, wherein haloalkyl is as defined above. Unless otherwise stated specifically in the specification, a haloalkoxy group is optionally substituted.

“Hydroxylalkyl” or “hydroxyalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxyalkyl group is optionally substituted.

“Aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The aryl group may be substituted or unsubstituted. When substituted, the aryl group is substituted with one or more substituents as this term is defined below, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, phenoxy, heteroaryl, heteroaryloxy, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heterocyclyl,” “heterocyclo,” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen and sulfur, and at least one aromatic ring. Heteroaryl radicals may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted.

“Fused ring” refers to a polycyclic ring systems (i.e., more than two cyclic rings) in which any two adjacent rings have at least two adjacent atoms in common. Fused ring may be a monocyclic, bicyclic, tricyclic or tetracyclic ring system which may include heteroatoms. Examples include, but are not limited to, octahydrocyclopenta[c]pyrrole, hexahydrocyclopenta[c]pyrrol-1(2H)-one, 3,6-dihydrocyclopenta[c]pyrrol-1(2H)-one, 4,5-dihydropyrrolo[3,4-b]pyrrol-6(1H)-one, 4,5-dihydro-6H-furo[2,3-c]pyrrol-6-one, 3,4-dihydrocyclopenta[c]pyrrol-1(2H)-one, 5,6-dihydropyrrolo[3,4-b]pyrrol-4(1H)-one, 5,6-dihydro-4H-furo[2,3-c]pyrrol-4-one, isoindolin-1-one, 3,8-dihydroindeno[1,2-c]pyrrol-1(2H)-one, 1,4-dihydropyrrolo[3,4-b]indol-3(2H)-one, 1,2-dihydro-3H-benzofuro[2,3-c]pyrrol-3-one, or the like.

“Substituent” is one or a group of atoms that replaces (one or more) atoms, thereby becoming a moiety in the resultant (new) molecule. For example, the one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof.

The term “substituted” used herein means any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro or bromo. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

“Prodrug” indicates a compound that may be converted under physiological conditions or by solvolysis to a biologically active compound of the disclosure. Thus, the term “prodrug” refers to a metabolic precursor of a compound of the disclosure that is pharmaceutically acceptable. A prodrug may be inactive when administered to a subject in need thereof, but is converted in vivo to an active compound of the disclosure. Prodrugs are typically rapidly transformed in vivo to yield the parent compound of the disclosure, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the disclosure in vivo when such prodrug is administered to a mammalian subject. Prodrugs of a compound of the disclosure may be prepared by modifying functional groups present in the compound of the disclosure in such a way that the modifications are cleaved, either in routine manipulation or in vivo, to the parent compound of the disclosure. Prodrugs include compounds of the disclosure wherein a hydroxy, amino, or mercapto group is bonded to any group that, when the prodrug of the compound of the disclosure is administered to a mammalian subject, cleaves to form a free hydroxy, free amino or free mercapto group, respectively. Examples of prodrugs include, but are not limited to, acetate, formate, and benzoate derivatives of alcohol or amide derivatives of amine functional groups in the compounds of the disclosure and the like.

The embodiments disclosed herein is also meant to encompass all pharmaceutically acceptable compounds of the compound of Structures (I)-(Ih) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled compounds of Structures (I)-(Ih), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Structures (I)-(Ih) can generally be prepared by conventional techniques known to those skilled in the art or by processes analogous to those described in the Preparations and Examples as set out below using an appropriate isotopically-labeled reagent in place of the non-labeled reagent previously employed.

The embodiments disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabelled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid (TFA), undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of the disclosure which, when administered to a mammal, preferably a human, is sufficient to effect treatment in the mammal, preferably a human. The amount of a lipid nanoparticle of the disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the disclosure, or their salt (e.g., pharmaceutically acceptable salt), may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another. The present disclosure also contemplates “diastereomers”, which refers to non-mirror image of non-identical stereoisomers. Diastereomers occur when two or more stereoisomers of a compound have different configurations at one or more of the equivalent stereocenters and are not mirror images of each other.

A “tautomer” refers to a proton shift from one atom of a molecule to another atom of the same molecule. The present disclosure includes tautomers of any said compounds.

### Pharmaceutical Compositions

Other embodiments are directed to pharmaceutical compositions. The pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the compound is delivered in a targeted drug delivery system, for example, in a liposome coated with and organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In treatment methods according to embodiments of the disclosure, an effective amount of at least one compound of Structures (I)-(Ih) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structures (I)-(Ih), and a pharmaceutically acceptable carrier.

Provided herein are pharmaceutical compositions comprising one or more compounds selected from compounds of Structures (I)-(Ih) and pharmaceutically acceptable diluent(s), excipient(s), and carrier(s). In certain embodiments, the compounds described are administered as pharmaceutical compositions in which one or more compounds selected from compounds of Structures (I)-(Ih) are mixed with other active ingredients, as in combination therapy. Encompassed herein are all combinations of actives set forth in the combination therapies section below and throughout this disclosure. In specific embodiments, the pharmaceutical compositions include one or more compounds of Structures (I)-(Ih).

A pharmaceutical composition, as used herein, refers to a mixture of one or more compounds selected from compounds of Structures (I)-(Ih) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structures (I)-(Ih) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structures (I)-(Ih) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structures (I)-(Ih) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water soluble form. In additional embodiments, suspensions of one or more compounds selected from compounds of Structures (I)-(Ih) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent or excipient, and one or more compounds selected from compounds of Structures (I)-(Ih), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structures (I)-(Ih) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structures (I)-(Ih). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained release system, such as semipermeable matrices of solid hydrophobic polymers containing the therapeutic agent. Various sustained release materials are useful herein. In some embodiments, sustained release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of one or more compounds selected from compounds of Structures (I)-(Ih) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structures (I)-(Ih) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structures (I)-(Ih) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structures (I)-(Ih) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

As mentioned above, the compounds and compositions of the disclosure will find utility in a broad range of diseases and conditions mediated by protein kinases, including diseases and conditions mediated by kinase. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

In some embodiments, a pharmaceutical composition has a compound described above and a pharmaceutically acceptable carrier including, for example, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, a method treating a disease or disorder, the method includes administering an effective amount of the compound or the pharmaceutical composition described herein to a subject in need thereof. In some embodiments, the subject is experiencing aberrant PAR₂ activity. In some embodiments, the subject is at risk for experiencing aberrant PAR₂ activity. In some embodiments, the subject is mammal. In some embodiments, the mammal is a human. In some embodiments, the subject has or is at risk for developing an inflammatory condition involving aberrant PAR₂ activity. In some embodiments, the disease or disorder is asthma, migraine related pain, chronic pain, cancer, and a vascular disorder.

In some embodiments, the method further comprises administering to the subject one or more anti-inflammatory agents. In some embodiments, the anti-inflammatory agent is a non-steroidal anti-inflammatory drug.

In some embodiments, a kit comprises a pharmaceutical composition, a container, pack, or dispenser, and instructions for administration.

### Preparation of Compounds

Preparation methods for the above compounds and compositions are described herein below and/or known in the art. It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, trityl, t-butyl, and the like. Suitable protecting groups for amino, amidino and guanidino include t-butoxycarbonyl, benzyloxycarbonyl, fluorenylmethyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs”. All prodrugs of compounds of this disclosure are included within the scope of the disclosure.

Furthermore, all compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

The following Reaction Scheme illustrates methods to make compounds of this disclosure, i.e., compounds of Structure (I):

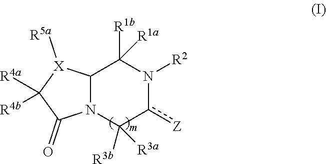

or a salt (e.g., pharmaceutically acceptable salt) or stereoisomer thereof, wherein R^{1a}, R^{1b}, R², R^{3a}, R^{3b}, R^{4a}, R^{4b}, R^{5a}, Z, X, and m are as defined herein. It is understood that one skilled in the art may be able to make these compounds by similar methods or by combining other methods known to one skilled in the art. It is also understood that one skilled in the art would be able to make, in a similar manner as described below, other compounds of Structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this disclosure.

### Embodiments

The disclosure further provides the following embodiments:

Embodiment 1. A compound having the following Structure (I):

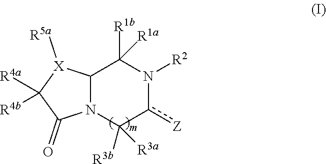

or a stereoisomer or salt thereof, wherein: R^{1a} and R^{1b} are, each independently, hydrogen, C₁-C₆ alkyl, C₁-C₆ heteroalkyl, C₃-C₈ cycloalkyl, aryl, or arylalkyl; R² is hydrogen, C₁-C₆ alkyl, C₁-C₆ heteroalkyl, C₃-C₈ heterocyclylalkyl, C₃-C₈ heterocyclylalkylC₁₋₆alkyl, heteroaryl, heteroarylalkyl, or arylalkyl; Z is either i) oxygen when [symbol] is a double bond or ii) hydrogen, C₁-C₆ alkyl, C₁-C₆ heteroalkyl, C₃-C₈ cycloalkyl, or arylalkyl when [symbol] is a single bond; R^{3a} and R^{3b} are, each independently, hydrogen, C₁-C₆ alkyl, C₁-C₆ heteroalkyl, C₃-C₈ cycloalkyl, or arylalkyl; X is N or C(R^{5b}); either i) R^{4a} and R^{4b} are, each independently, hydrogen, C₁-C₆ alkyl, C₁-C₆ heteroalkyl, C₃-C₈ cycloalkyl, C₃-C₈ heterocyclylalkyl, arylalkyl, or heteroarylalkyl; and R^{5a} and R^{5b} are, each independently, hydrogen, C₁-C₆ alkyl, C₁-C₆ hydroxyalkyl, C₃-C₈ cycloalkyl, C₃-C₈ heterocyclylalkyl, arylalkyl, heteroarylalkyl, —C(═O)R⁶, —C(═O)YR⁶, —OC(═O)R⁶, —OC(═O)YR⁶, —NHC(═O)R⁶, or —NHC(═O)YR⁶, provided that when R^{5a} and R^{5b} are, each independently, —NHC(═O)R⁶, or —NHC(═O)YR⁶, R⁶ is not an arylalkyl; or ii) R^{4a} forms a direct bond with X and R^{4b} and R^{5b} together form a 5-9 member fused ring; R⁶ is C₃-C₈ cycloalkyl, C₃-C₈ heterocyclylalkyl, arylalkyl, heteroarylalkyl, or a 5-9 member fused ring; Y is O, S, or NR⁷; R⁷ is hydrogen, hydroxyl, or C₁-C₆ alkyl; m is 1, or 2; and wherein each of R^{1a}, R^{1b}, R², R^{3a}, R^{3b}, R^{4a}, R^{4b}, R^{5a}, R^{5b}, R⁶, and R⁷ is optionally substituted with one or more substituent.

Embodiment 2. The compound of embodiment 1, wherein m is 1.

Embodiment 3. The compound of embodiment 1 or embodiment 2, wherein X is N.

Embodiment 4. The compound of embodiment 3 having the following Structure (Ia):

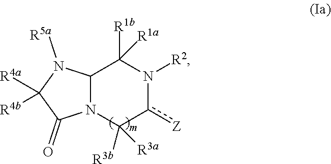

or a stereoisomer or salt thereof.

Embodiment 5. The compound of any one of embodiments 1-4, wherein Z is oxygen.

Embodiment 6. The compound of embodiment 5 having the following Structure (Ib):

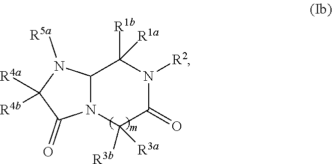

or a stereoisomer or salt thereof.

Embodiment 7. The compound of embodiment 1, wherein m is 2.

Embodiment 8. The compound of embodiment 7, wherein X is N.

Embodiment 9. The compound of embodiment 8 having the following Structure (Ic):

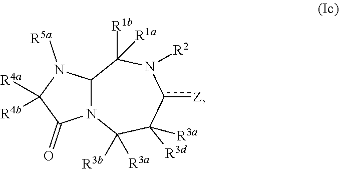

or a stereoisomer or salt thereof.

Embodiment 10. The compound of embodiment 9, wherein Z is an oxygen.

Embodiment 11. The compound of embodiment 10 having the following Structure (Id):

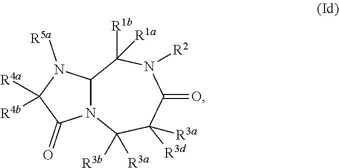

or a stereoisomer or salt thereof.

Embodiment 12. The compound of embodiment 1, wherein the compound has one of the following Structures (Ia)-(Id):

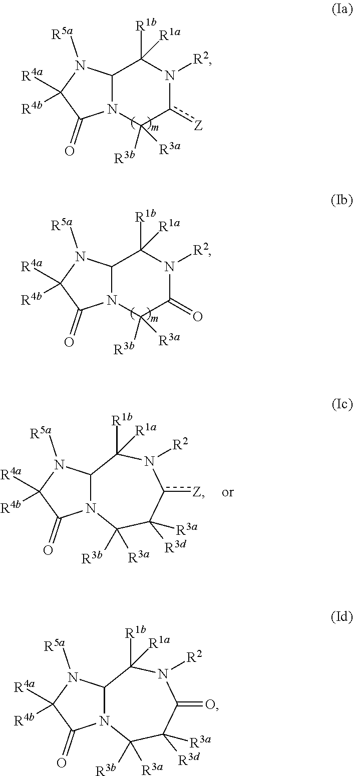

or a stereoisomer or salt thereof.

Embodiment 13. The compound of any one of embodiments 1-12, wherein X is C(R^{5b}).

Embodiment 14. The compound of any one of embodiments 1-13, wherein R^{4a} forms a direct bond with X and R^{4b} and R^{5b} together form a 5-9 member fused ring.

Embodiment 15. The compound of embodiment 14, wherein the compound has one of the following Structures (Ie)-(Ih):

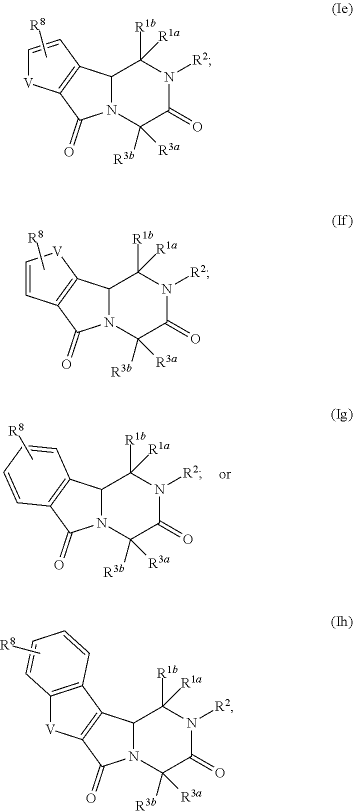

or a stereoisomer or salt thereof, wherein R⁸ is hydrogen, halo, hydroxyl, alkoxy, amino, cyano, C₁-C₆ alkyl, or C₁-C₆ heteroalkyl; and V is a carbon, nitrogen, or oxygen atom.

Embodiment 16. The compound of any one of embodiments 1-15, wherein at least one of R^{1a} or R^{1b} is hydrogen.

Embodiment 17. The compound of any one of embodiments 1-15, wherein each of R^{1a} and R^{1b} are hydrogen.

Embodiment 18. The compound of any one of embodiments 1-15, wherein R^{1a} and R^{1b} have one of the following structures:

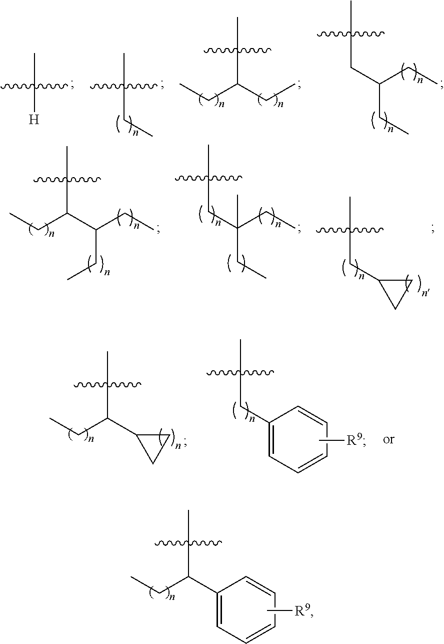

wherein n is, each independently, 0-6 and n′ is 1-5; and R9 is hydrogen, halo, hydroxyl, alkoxy, amino, cyano, C1-C6 alkyl, or C1-C6 heteroalkyl.

Embodiment 19. The compound of any one of embodiments 1-18, wherein R² has one of the following structures:

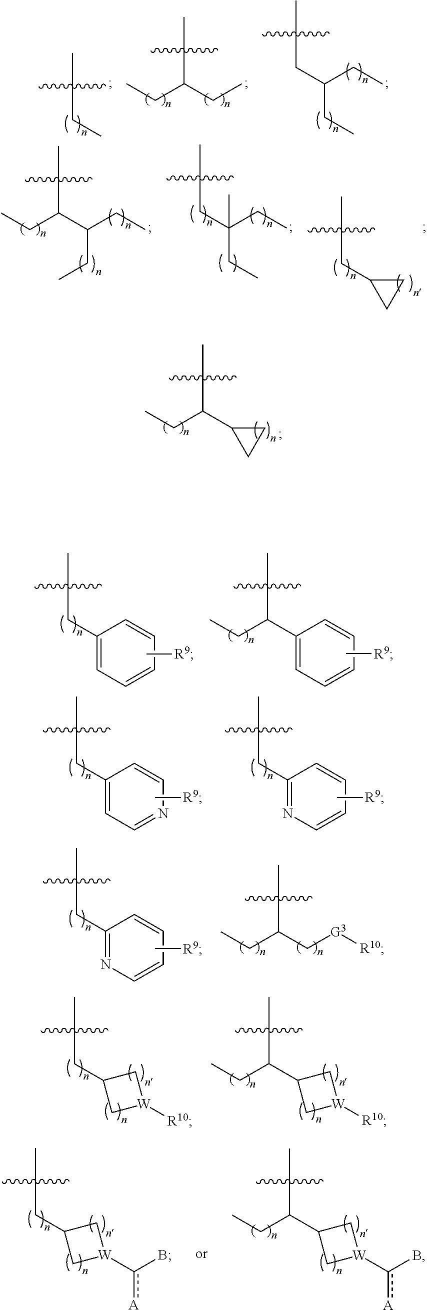

wherein n is, each independently 0-6 and n′ is 1-5; G² is CH₂, NH, O or S; W is CH or N; A is O, NH, or NHR⁹, wherein i) [symbol] is a double bond when A is O or ii) [symbol] is a single bond when A is NH or NHR⁹; B is OR⁹, NH₂, or NHR⁹; R⁹ is hydrogen, halo, hydroxyl, alkoxy, amino, cyano, C₁-C₆ alkyl, or C₁-C₆ heteroalkyl; and R¹⁰ is either i) absent or ii) hydrogen, alkyl, aryl, pyridyl, acylalkyl, or acylaryl.

Embodiment 20. The compound of any one of embodiments 1-19, wherein R² is

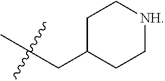 [SMILES: *CC1CCNCC1]

Embodiment 21. The compound of any one of embodiments 1-20, wherein R^{3a} or R^{3b} has one of the following structures:

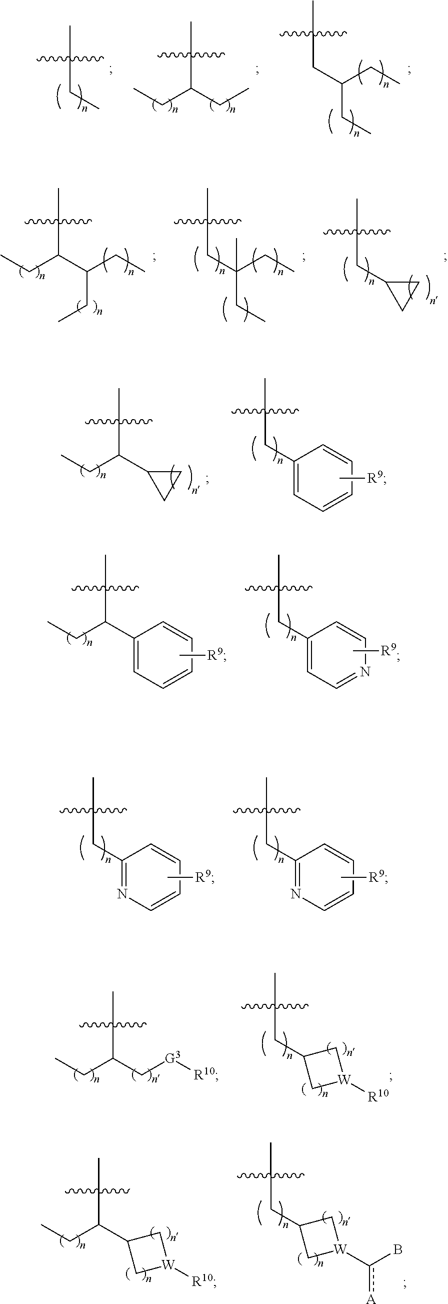

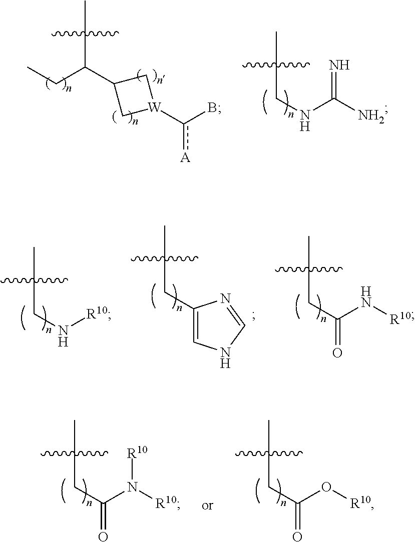

wherein n is, each independently, 0-6 and n′ is 1-5; G3 is, each independently C(R7)2, O, S, or NR7; W is, each independently, CH or N; A is O, NH, or NHR9, wherein i) [symbol] is a double bond when A is O or ii) [symbol] is a single bond when A is NH or NHR9; B is OR9, NH2, or NHR9; R9 is hydrogen, halo, hydroxyl, alkoxy, amino, cyano, C1-C6 alkyl, or C1-C6 heteroalkyl; and R10 is either i) absent or ii) hydrogen, alkyl, aryl, pyridyl, acylalkyl, or acylaryl.

Embodiment 22. The compound of any one of embodiments 1-21, wherein one of R^{3a} or R^{3b} is hydrogen and the other one of R^{3a} or R^{3b} is

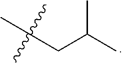 [SMILES: *CC(C)C]

Embodiment 23. The compound of any one of embodiments 1-22, wherein one of R^{3a} or R^{3b} is hydrogen and the other one of R^{3a} or R^{3b} is

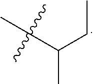 [SMILES: *C(C)CC]

Embodiment 24. The compound of any one of embodiments 1-23, wherein R^{4a} or R^{4b} has one of the following structures:

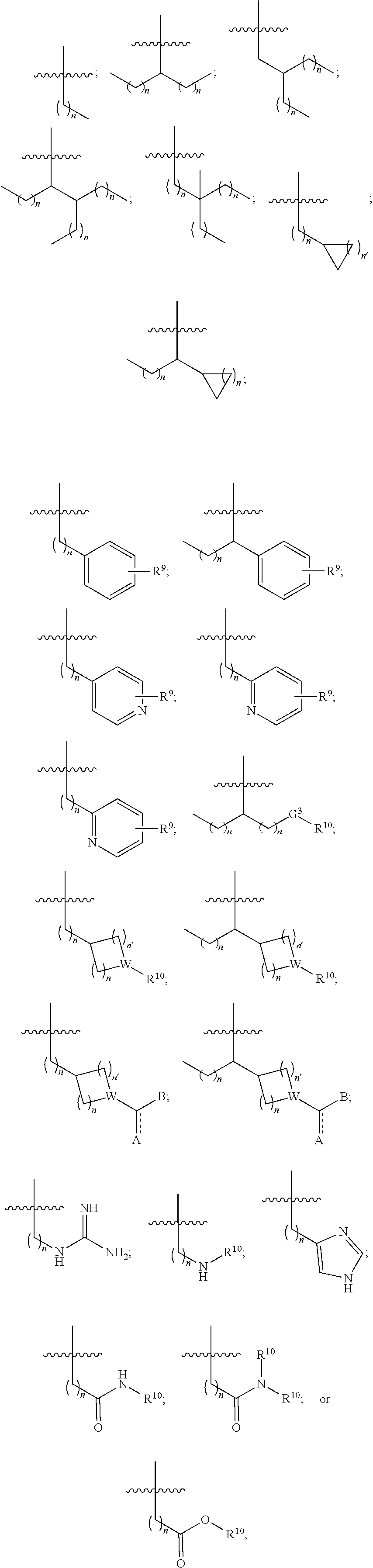

wherein n is, each independently, 0-6 and n′ is 1-5; G4 is, each independently, C(R7)2, O, S, or NR7; W is, each independently, CH or N; A is O, NH, or NHR9, wherein i) [symbol] is a double bond when A is O or ii) [symbol] is a single bond when A is NH or NHR9; B is OR9, NH2, or NHR9; R9 is hydrogen, halo, hydroxyl, alkoxy, amino, cyano, C1-C6 alkyl, or C1-C6 heteroalkyl; and R10 is either i) absent or ii) hydrogen, alkyl, aryl, pyridyl, acylalkyl, or acylaryl.

Embodiment 25. The compound of any one of embodiments 1-24, wherein one of R^{4a} or R^{4b} is hydrogen and the other one of R^{4a} or R^{4b} is

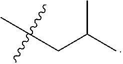 [SMILES: *CC(C)C]

Embodiment 26. The compound of any one of embodiments 1-25, wherein R^{5a} has one of the following structures:

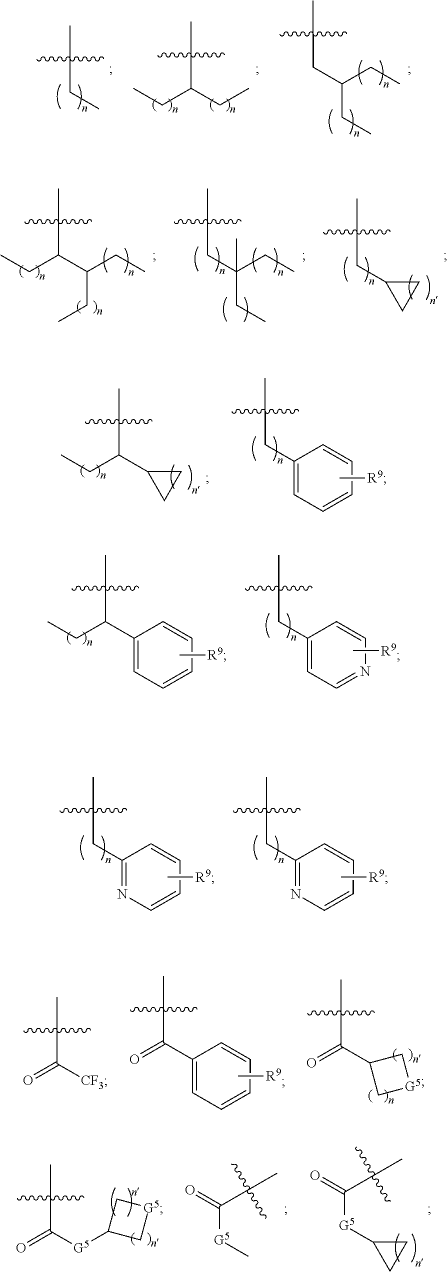

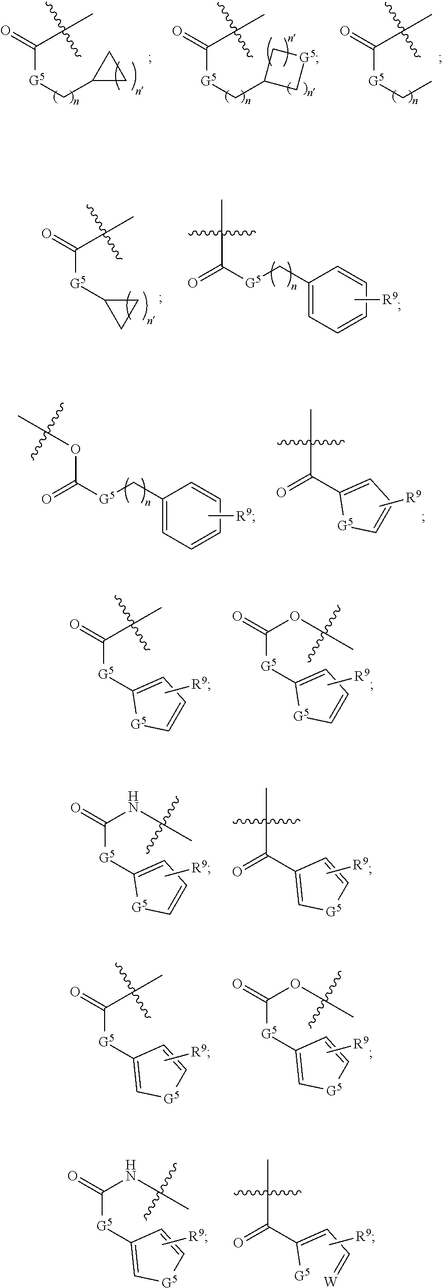

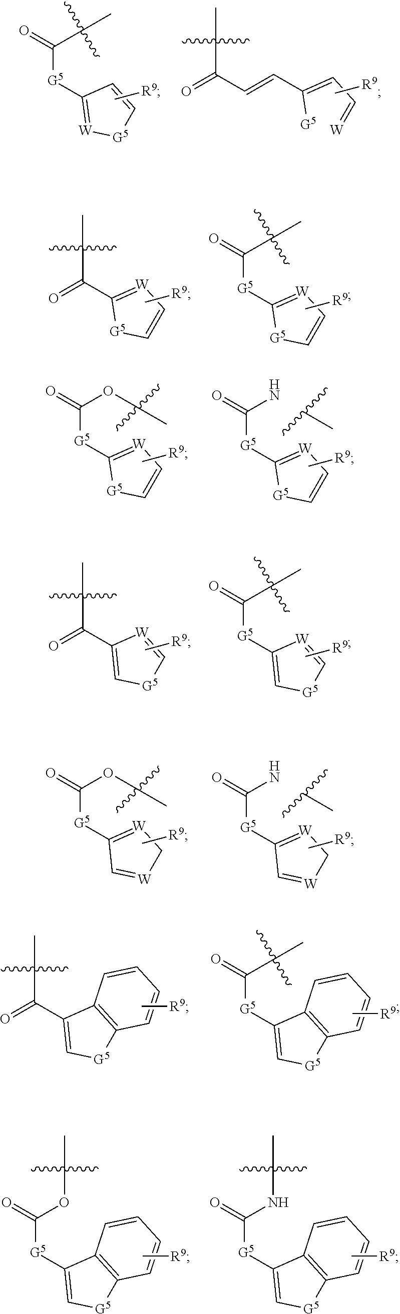

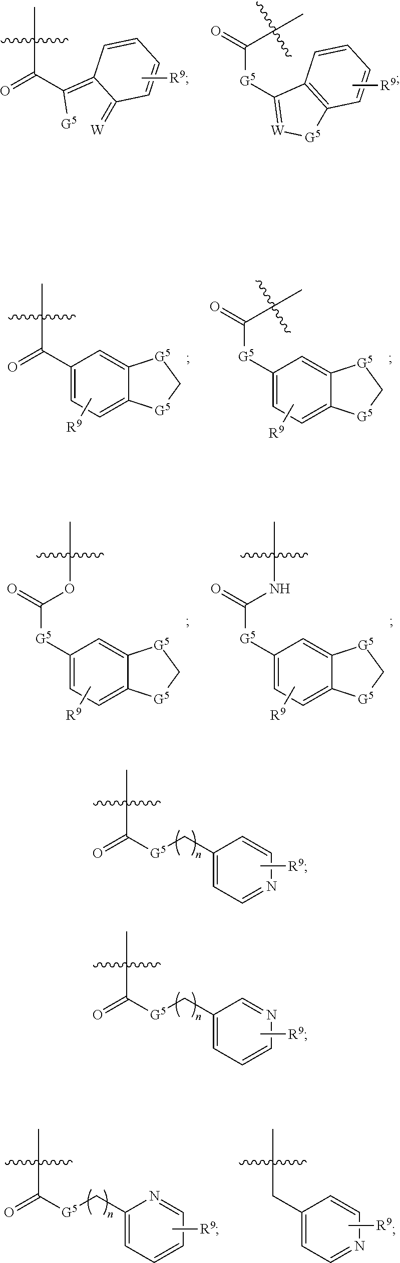

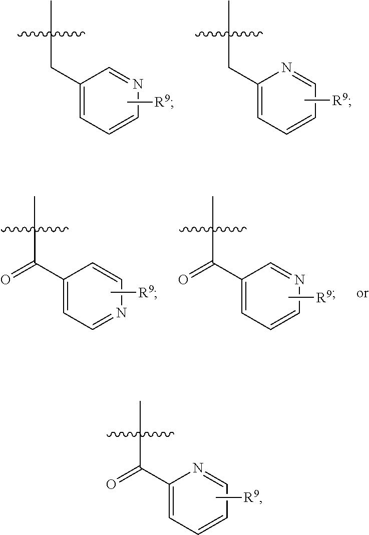

wherein n is 0-6 and n′ is 1-5; G5 is, each independently, C(R7)2, O, S, or NR7; and W is, each independently, CH or N.

Embodiment 27. The compound of any one of embodiments 1-26, wherein R^{5a} is

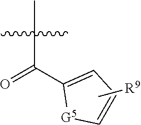

and R^{5b} is hydrogen.

Embodiment 28. The compound of embodiment 27, wherein W is O and R⁹ is hydrogen.

Embodiment 29. The compound of any one of embodiments 1-25, wherein R^{5a} has one of the following structures:

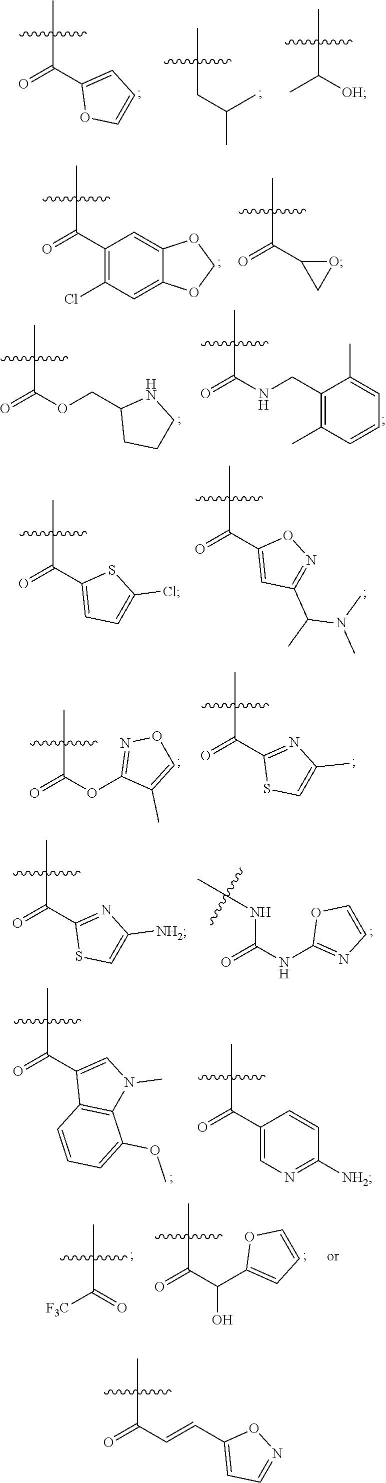

Embodiment 30. The compound of embodiment 1, wherein the compound has one of the following structures:

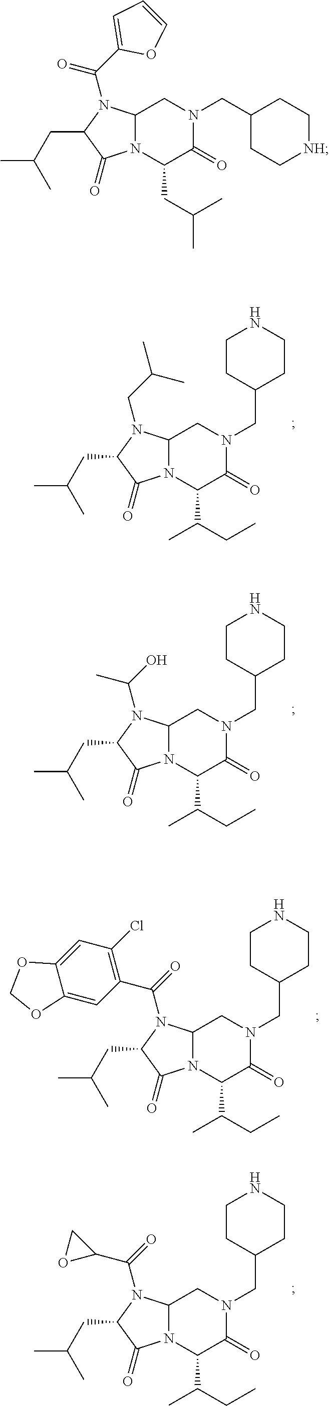 [SMILES: CC(C)CC1C(=O)N2C(CN(CC3CCNCC3)C(=O)[C@@H]2CC(C)C)N1C(=O)c1ccco1 ; CCC(C)[C@H]1C(=O)N(CC2CCNCC2)CC2N(CC(C)C)[C@@H](CC(C)C)C(=O)N21 ; CCC(C)[C@H]1C(=O)N(CC2CCNCC2)CC2N1C(=O)[C@H](CC(C)C)N2C(C)O ; CCC(C)[C@H]1C(=O)N(CC2CCNCC2)CC2N1C(=O)[C@H](CC(C)C)N2C(=O)c1cc2c(cc1Cl)OCO2 ; CCC(C)[C@H]1C(=O)N(CC2CCNCC2)CC2N1C(=O)[C@H](CC(C)C)N2C(=O)C1CO1]

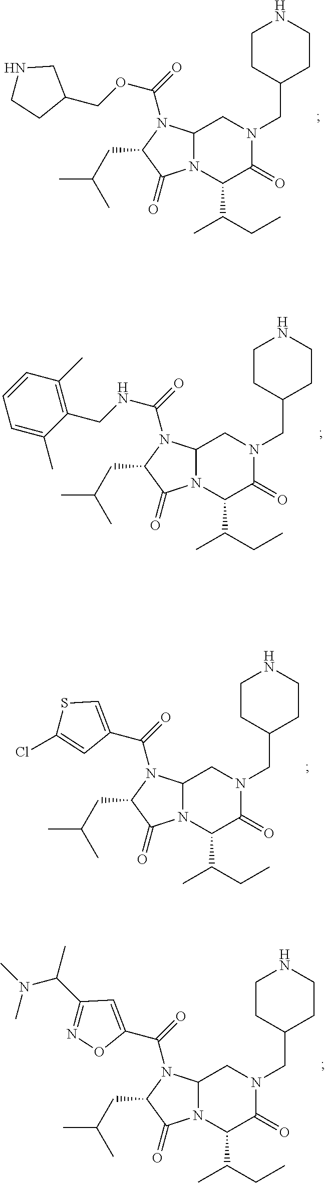 [SMILES: CCC(C)[C@H]1C(=O)N(CC2CCNCC2)CC2N1C(=O)[C@H](CC(C)C)N2C(=O)OCC1CCNC1 ; CCC(C)[C@H]1C(=O)N(CC2CCNCC2)CC2N1C(=O)[C@H](CC(C)C)N2C(=O)NCc1c(C)cccc1C ; CCC(C)[C@H]1C(=O)N(CC2CCNCC2)CC2N1C(=O)[C@H](CC(C)C)N2C(=O)c1csc(Cl)c1 ; CCC(C)[C@H]1C(=O)N(CC2CCNCC2)CC2N1C(=O)[C@H](CC(C)C)N2C(=O)c1cc(C(C)N(C)C)no1]

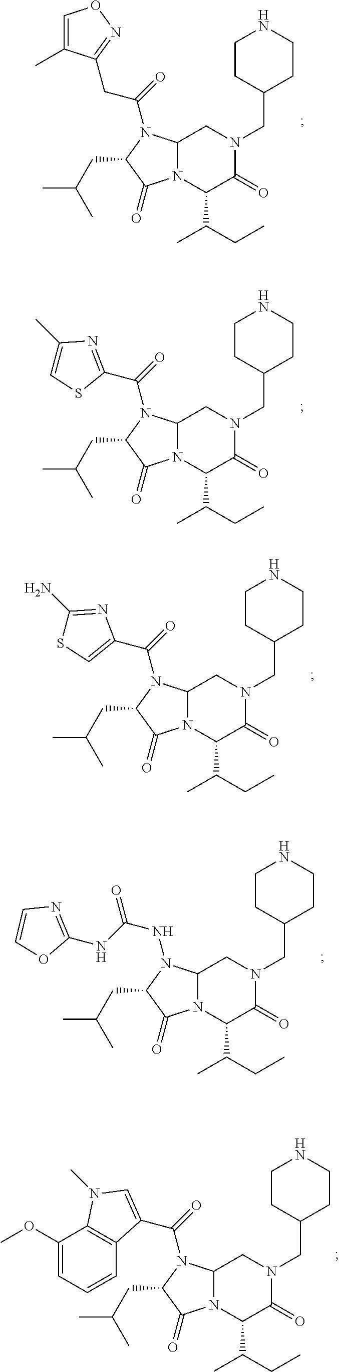 [SMILES: CCC(C)[C@H]1C(=O)N(CC2CCNCC2)CC2N1C(=O)[C@H](CC(C)C)N2C(=O)Cc1nocc1C ; CCC(C)[C@H]1C(=O)N(CC2CCNCC2)CC2N1C(=O)[C@H](CC(C)C)N2C(=O)c1nc(C)cs1 ; CCC(C)[C@H]1C(=O)N(CC2CCNCC2)CC2N1C(=O)[C@H](CC(C)C)N2C(=O)c1csc(N)n1 ; CCC(C)[C@H]1C(=O)N(CC2CCNCC2)CC2N(NC(=O)Nc3ncco3)[C@@H](CC(C)C)C(=O)N21 ; CCC(C)[C@H]1C(=O)N(CC2CCNCC2)CC2N1C(=O)[C@H](CC(C)C)N2C(=O)c1cn(C)c2c(OC)cccc12]

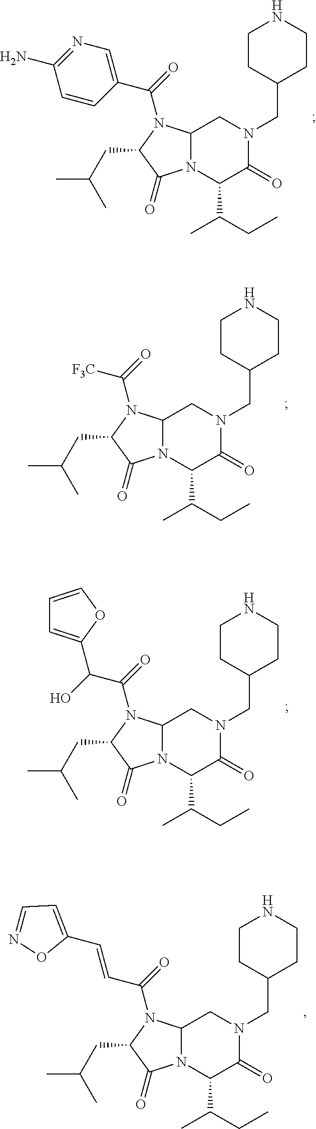 [SMILES: CCC(C)[C@H]1C(=O)N(CC2CCNCC2)CC2N1C(=O)[C@H](CC(C)C)N2C(=O)c1ccc(N)nc1 ; CCC(C)[C@H]1C(=O)N(CC2CCNCC2)CC2N1C(=O)[C@H](CC(C)C)N2C(=O)C(F)(F)F ; CCC(C)[C@H]1C(=O)N(CC2CCNCC2)CC2N1C(=O)[C@H](CC(C)C)N2C(=O)C(O)c1ccco1 ; CCC(C)[C@H]1C(=O)N(CC2CCNCC2)CC2N1C(=O)[C@H](CC(C)C)N2C(=O)/C=C/c1ccno1]

or a stereoisomer or salt thereof.

Embodiment 31. The compound of any one of embodiments 1-30, wherein the compound is a pharmaceutically acceptable salt.

Embodiment 32. A pharmaceutical composition comprising a compound or salt of any one of embodiments 1-31 and a pharmaceutically acceptable carrier.

Embodiment 33. A method of treating a disease or disorder, the method comprising administering an effective amount of the compound of any one of embodiments 1-30 or the pharmaceutical composition of embodiment 32 to a subject in need thereof.

Embodiment 34. The composition of any one of embodiments 1-30 or the pharmaceutical composition of embodiment 32 for use in a method of treating a disease or disorder.

Embodiment 35. The composition of any one of embodiments 1-30 or the pharmaceutical composition of embodiment 32 for use in the preparation of a medicament for the treatment of a disease or disorder.

Embodiment 36. The method of embodiment 33 or the composition of embodiment 34 or 35, wherein the subject is experiencing aberrant PAR₂ activity involved in the disease or disorder.

Embodiment 37. The method of embodiment 33 or the composition of embodiment 34 or 35, wherein the subject is at risk for experiencing aberrant PAR₂ activity involved in the disease or disorder.

Embodiment 38. The method of embodiment 33 or the composition of embodiment 34 or 35, wherein the disease or disorder is in a mammal.

Embodiment 39. The method or composition of embodiment 38, wherein the mammal is a human.

Embodiment 40. The method or composition of embodiment 39, wherein the mammal has or is at risk for developing an inflammatory condition involving aberrant PAR₂ activity.

Embodiment 42. The method or composition of embodiment 40, wherein the inflammatory condition is one or more conditions selected from the group consisting of asthma, migraine related pain, chronic pain, cancer, and a vascular disorder.

Embodiment 43. The method or composition of embodiment 40, further comprising administering to the subject one or more anti-inflammatory agents or the composition of claim 32 further comprising one or more anti-inflammatory agents.

Embodiment 44. The method or composition of embodiment 43, wherein the anti-inflammatory agent is a non-steroidal anti-inflammatory drug.

Embodiment 45. A kit comprising a pharmaceutical composition of embodiment 32, a container, pack, or dispenser, and instructions for administration.

Embodiment 46. The kit of embodiment 45, wherein the instructions for administration comprise the method or use of any one of embodiments 33-44.

### Examples

The following examples are provided for purpose of illustration and not limitation.

Compound I-1 as used in these Examples is 4-(((2R,5S)-1-(furan-2-carbonyl)-2,5-diisobutyl-3,6-dioxohexahydroimidazolo [1,2-a]pyrazin-7(1H)-yl)methyl)piperidin-1-ium chloride.

### Example 1: Preparation of Compound I-1

Abbreviations: The following abbreviations are used throughout this Example 1.

° C. (degree Celsius); ¹H NMR (proton Nuclear Magnetic Resonance); DCM (dichloromethane); DMSO (dimethylsulfoxide); eq (equivalent); EtOAc (ethyl acetate); g (gram); h (hour); MeOH (methanol); mg (milligram); min (minute); mL (milliliter); mmol (millimole); TFA (trifluoroacetic acid); THE (tetrahydrofuran); TLC (Thin Layer Chromatography); Boc (t-butyloxycarbonyl); DIC (N,N′-diisopropylcarbodiimide); DIEA (diisopropylethylamine); DMF (N,N′-dimethylformamide); EDT (1,2-ethylene dithiol); ESI-MS (electrospray ionization-mass spectrometry); Et₂O (diethylether); Fmoc ((9H-fluoren-9-ylmethoxy)carbonyl); HBTU (2-(1H-benzotriazol-1-yl)-1,1,3,3-tetramethyluronium hexafluorophosphate); HOBt (N-hydroxybenzotriazole); HPLC (high performance liquid chromatography); OBt (O-benzotriazolyl); Pd(TPP)₄ (Palladium (0) tetrakistriphenylphoshine); SPE (solid-phase extraction); SPPS (solid-phase peptide synthesis); TCFH (tetramethylchloroformamidium hexafluorophosphate); and TMS (tetramethylsilane).

Chemical Materials: The following reagents and materials are used throughout this Example 1.

N^{α}-Fmoc-protected amino acids, activating agents (HBTU, TCFH, DIC, and HOBt) were purchased from P3BioSystems (Louisville, KY) or from Novabiochem (San Diego, CA). Bromoacetal and 2-Chloro-chlorotrityl resins were acquired from Rapp Polymere (Tubingen, Germany). Reagent grade solvents, reagents, and acetonitrile for HPLC were acquired from VWR (West Chester, PA) or Sigma-Aldrich (St. Louis, MO) and were used without further purification unless otherwise noted. Chemicals and reagents were obtained from Sigma-Aldrich or TCI (Portland, OR). The solid-phase synthesis was performed in fritted syringes using a Domino manual synthesizer obtained from Torviq (Tucson, AZ).

### General Synthesis:

1H NMR spectra were recorded on a Bruker-DRX-300 MHz instrument with chemical shifts reported relative to tetramethylsilane internal standard (TMS, 0.0 ppm) and residual dimethyl sulfoxide (DMSO; 2.50 ppm). Proton-decoupled ¹³C NMR spectra were referenced to CDCl₃ (77.0 ppm) as well as DMSO (39.51 ppm). Low resolution mass spectra were obtained on a Bruker AmaZon SL Ion Trap Mass Spectrometer with ESI source (Madison, WI). High resolution mass spectra (HRMS) were recorded on a Bruker SolariX Fourier-transform Ion Cyclotron Resonance Mass Spectrometer, 9.4T magnet, with ESI source (Madison, WI).

### Quality Control and Purification:

The purity of products was checked by analytical reverse phase HPLC using an Agilent 1260 Infinity II Separation Model (Santa Clara, CA) with dual wavelength detector (220 and 280 nm) on a reverse phase column (Agilent Boroshell C18, 3.0×100 mm, 2.7 μm). Crude compounds were eluted with a linear gradient of aqueous CH₃CN/0.1% CF₃CO₂H at a flow rate of 0.3 mL/min. Purification of compounds was achieved on an Agilent 1260 Infinity II Prep Model HPLC using a reverse phase column (YMC ODS C18, 10 μm, 20×250 mm). Compounds were eluted with a linear gradient of CH₃CN/0.1% CF₃CO₂H at a flow rate of 3.0-10.0 mL/min. Separation was monitored at suitable wavelength in range 220-280 nm. Flash chromatography was performed on a borosilicate glass column (2.6×250 mm, Sigma-Aldrich) filled with 60 Å silicagel (Sigma-Aldrich). The compounds were eluted with a step gradient flow of hexane/EtOAc/MeOH mixtures.

### Preparation of the Compound I-1 Salt

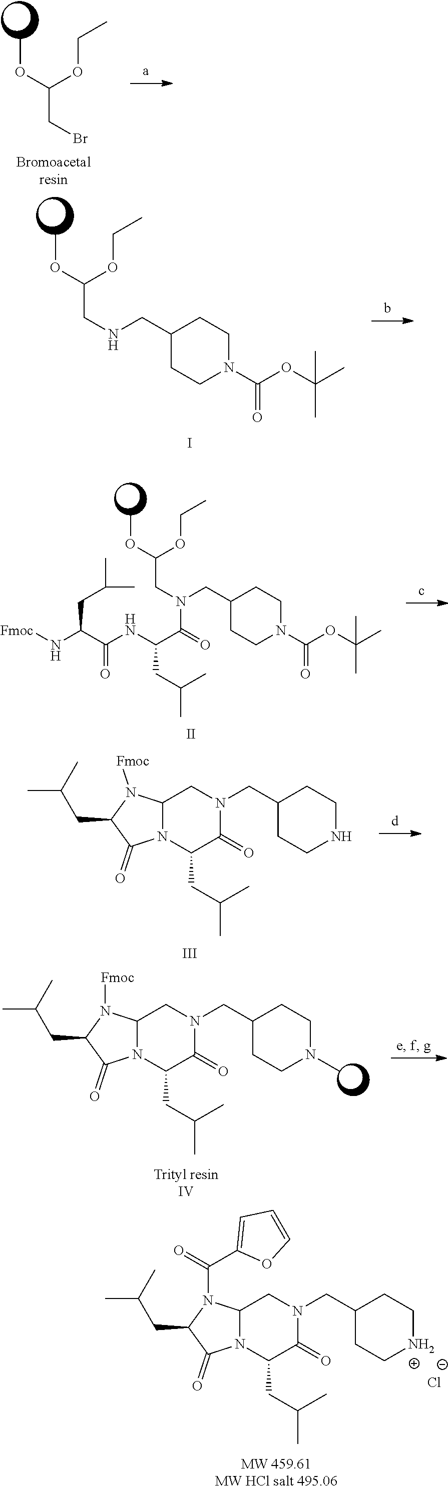

### Solid-Phase Synthesis of Compound I-1

(a) 0.5M solution of Boc-protected diamine in DMSO, 60° C., overnight. (b) (i) Fmoc-Leu coupling, TCFH/collidine (II) 10% piperidine in DMF, 20 minutes (iii) Fmoc-Leu, DIC/HOBt, 3 hrs. (c) (i) 60% a. formic acid, 4 hrs (ii) column purification. (d) attachment to the 2-Chloro-chlorotrityl resin, 2.5 eq. in DCM, DIEA. (e) 10% piperidine in DMF, 20 minutes (f) 0.5 M furoic acid, DCM, TCFH, DIEA, overnight. (g) (i) 94% TFA in DCM, 3% water, 1 hr. (ii) HPLC purification.

Intermediate I: The bromoacetal resin (0.2 g, 0.25 mmol) was swelled for 2 hours by the addition of DCM. The swollen resin was carried out through the solid-phase procedure. The resin was washed successively with DMF (3×2 min), DCM (3×2 min), DMSO (5×2 min). Amine displacement of bromine was accomplished with 0.5M solution of 1-N-Boc-4-(aminomethyl)piperidine (0.214 g, 1 mmol, 4 eq) in DMSO at 60° C. overnight. The reaction mixture was removed by filtration and the resin was washed successively with DMSO (5×2 min) and DCM (3×2 min). Before coupling of Fmoc-Leu, the resin was neutralized with 10% diisopropylethylamine (DIEA) in DCM (2×3 min), followed by washing successively with DCM (3×2 min) and DMF (5×2 min).

Intermediate II: The intermediate resin I from above (0.2 g, 0.25 mmol) was coupled with Fmoc-Leu. The coupling was performed by adding Fmoc-Leu (0.353 g, 1 mmol, 4 eq) and coupling reagents [TCFH (0.28 g, 1 mmol, 4 eq) and DIEA (0.35 mL, 2 mmol, 8 eq)] in DMF (2 mL) to the resin and stirring the mixture overnight. The second TCFH coupling was performed for 2 hour in THF/DMF mixture. The reaction mixture was removed by filtration and the resin was washed with DMF (5×2 min) and DCM (3×2 min). The Na-Fmoc protecting group was removed with 1:10 piperidine in DMF (1×2 min and 1×20 min). The resin was washed successively with DMF (5×2 min), DCM (3×2 min), a solution of 0.05 mM solution of Bromophenol Blue in 0.2 M HOBt in DMF, then DMF (3×2 min). The Na-Fmoc-Leu was coupled using pre-activated 0.3 M HOBt esters in DMF-DCM mixture (3 eq of Na-Fmoc-Leu, 3 eq of HOBt, and 3 eq of DIC). The resin slurry was stirred for 2 h or until the bromophenol test became negative (yellow). The resin was washed with DMF (3×2 min). A second coupling was performed by the HBTU/2,4,6-lutidine procedure (0.3 M solution of 3 eq of Na-Fmoc-Leu, 3 eq of HBTU, and 6 eq of 2,4,6-lutidine in DMF. The reaction mixture was removed by filtration and the resin was washed with DMF (5×2 min), DCM (3×2 min), and then MeOH (3×2 min). The reactor was placed in vacuum oven to remove solvents.

Intermediate III: The intermediate resin II from above (0.2 g, 0.25 mmol) was treated with 60% formic acid (2 mL) for 4 hrs. The resin was filtrated and washed with formic acid (2×2 min). Acid filtrate and washes were collected, diluted with DI water and lyophilized. The crude intermediate III was purified using column chromatography to yield pure III (74 mg, 53%, (M+H)+ 559.3, HPLC>95%).

Intermediate IV: The dry intermediate III from above (74 mg, 132 μmol) was added to dry 2-chloro-chlorotrityl resin (250 mg, 430 μmol, 2.5 eq.). Dry DCM (2.0 mL) and DIEA (171 μL, 1.0 mmol) were injected into the resin and the mixture was agitated at room temperature for overnight. The reaction was quenched by adding MeOH (0.2 mL, 30 min). The reaction mixture was removed by filtration and the resin was washed with DMF (3×2 min), DCM (5×2 min), and then DMF (2×2 min).

Final product compound I-1: The intermediate resin IV from above was deprotected by piperidine. The Na-Fmoc protecting group was removed with 1:10 piperidine in DMF (1×2 min and 1×20 min). The resin was washed with DMF (5×2 min) and then DCM (5×2 min). The 2-furoic acid was coupled using by adding 2-furoic acid (1 mmol, 4 eq) and coupling reagents [TCFH (0.28 g, 1 mmol, 4 eq) and DIEA (0.35 mL, 2 mmol, 8 eq)] in DMF (2 mL) to the resin and stirring the mixture overnight. The reaction mixture removed by filtration, the resin was washed with DCM (5×2 min) and the 2-furoic acid was coupled again using 5 eq in THF/DMF mixture. The reaction mixture was removed by filtration and the resin was washed successively with DMF (5×2 min), DCM (3×2 min). The reaction mixture was removed by filtration and the resin was washed successively with DMF (5×2 min), DCM (7×2 min), then cleaved by strong acid. A cleavage cocktail (2.0 mL) consisting of CF₃CO₂H (97%) and H₂O (3%) was injected into the resin and the mixture was agitated at room temperature for 1 h. The solution was filtered, the resin was washed with CF₃CO₂H (2×3 min), the liquid phases were collected and concentrated under a stream of nitrogen, and the product was precipitated using cold Et₂O/hexane. The crude product was washed three times with cold Et₂O/hexane, lyophilized, purified by HPLC, and characterized as described above. The desired product was 25 mg of compound I-1 as a white lyophilizate (yield 19%).

Analytical data of compound I-1: (M+H)+ 515.30, HPLC>95%).

¹H NMR: 0.86 (d, 6H), 0.90 (d, 6H), 1.30 (m, 4H), 1.60 (m, 1H), 1.74 (t, 2H), 1.97 (m, 1H), 2.0 (d, 2H), 2.02 (m, 1H), 2.81 (m, 4H), 3.3 (t, 2H), 3.50 (t, 2H), 3.70 (m, 2H), 4.5 (t, 1H), 5.0 (t, 1H), 6.10 (t, 1H), 6.7(t, 1H), 7.30 (d, 1H), 7.92 (d, 1H), 8.25 (d, 1H).

¹³C NMR: 26.8 (CH₃), 27.5 (CH₃), 28.3 (CH), 29.5 (CH), 30.7 (CH₂), 32.0 ((CH), 32.5 (CH), 48.2 ((CH₂), 49.5 (CH₂), 52.3 (CH₂), 52.8 (CH₂), 54.1 (CH₂), 58.5 (CH), 114.1 (CH), 117.3 (ArCH), 150.5 (ArCH), 151.5 (ArCH), 164.1 (CO), 170.07 (CO), 172.3 (CO), 177.6 (CO)

### Example 2: In Vitro Efficacy of Compound I-1

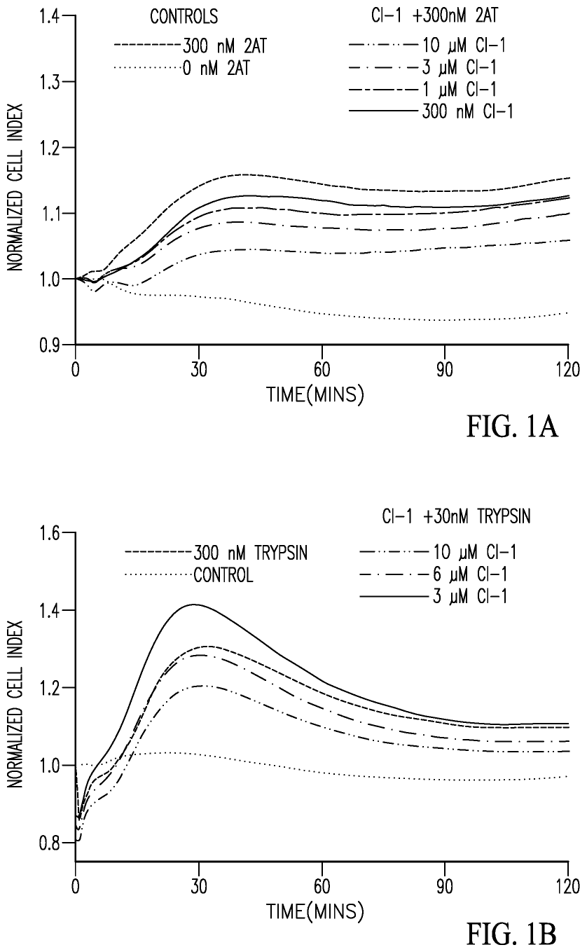

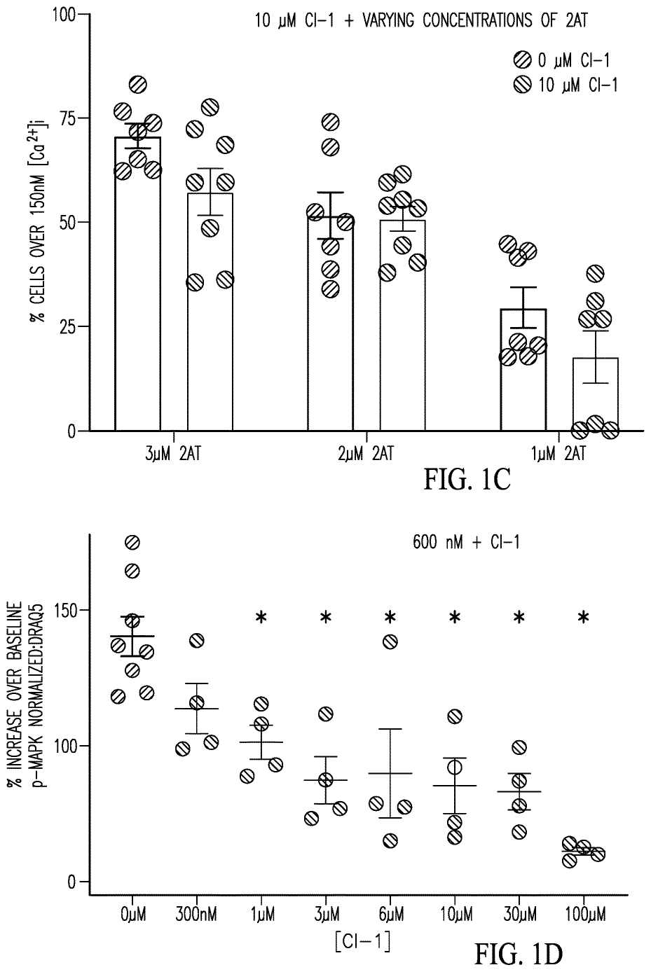

Compound I-1 (labelled as “C I-1” in FIGS. 1A-1D) was evaluated for its ability to antagonize protease-activated receptor-2 (PAR₂) in several cellular assays using a human bronchial epithelial cell line that naturally expressed PAR₂ (16HBE14o− cells; see FIG. 1A-1D).

In the first assay to detect an in vitro physiological response, the xCELLigence real time cellular analysis (RTCA) was used. The RTCA assay can identify full, partial and/or limited agonist potency as well as biased signaling and antagonism, see Boitano S, Flynn A N, Schulz S M, Hoffman J, Price T J, and Vagner J. “Potent Agonists of the Protease Activated Receptor 2 (PAR₂)”, J Med Chem (2011), 54, 1308-13; Boitano S, Hoffman J, Tillu D V, Asiedu M N, Zhang Z, Sherwood C L, Wang Y, Dong X, Price T J, and Vagner J. “Development and Evaluation of Small Peptidomimetic Ligands to Protease-Activated Receptor-2 (PAR₂) through the Use of Lipid Tethering”, PLoS One, (2014), 9, e99140; Flynn A N, Hoffman J, Tillu D V, Sherwood C L, Zhang Z, Patek R, Asiedu M N, Vagner J, Price T J, and Boitano S. “Development of highly potent protease-activated receptor 2 agonists via synthetic lipid tethering” Faseb J, (2013), 27, 1498-1510; Flynn A N, Tillu D V, Asiedu M N, Hoffman J, Vagner J, Price T J, and Boitano S. “The protease-activated receptor-2-specific agonists 2-aminothiazol-4-yl-LIGRL-NH₂ and 6-aminonicotinyl-LIGRL-NH₂ stimulate multiple signaling pathways to induce physiological responses in vitro and in vivo”, J Biol Chem, (2011), 286: 19076-88; and Rivas C M, Schiff H V, Moutul A, Khanna R, Kiela P R, Dussor G, Price T J, Vagner J, DeFea K A, and Boitano S. “Alternaria alternata-induced airway epithelial signaling and inflammatory responses via protease-activated receptor-2 expression”, Biochemical and Biophysical Research Communications, (2022).

Following overnight growth, varying concentrations of Compound I-1 was added to individual wells (in quadruplicate) in final concentrations of 300 nM, 1 μM, 3 μM and 10 μM. After a 5 min pre-incubation, a 300 nM concentration of the highly potent and specific PAR₂ antagonist 2-at-LIGRL-NH₂ [2AT, see Flynn A N, Tillu D V, Asiedu M N, Hoffman J, Vagner J, Price T J, and Boitano S. “The protease-activated receptor-2-specific agonists 2-aminothiazol-4-yl-LIGRL-NH₂ and 6-aminonicotinyl-LIGRL-NH₂ stimulate multiple signaling pathways to induce physiological responses in vitro and in vivo”, J Biol Chem, (2011), 286: 19076-88) or 30 nM trypsin was added to the wells, see FIGS. 1A and 1B. The upward deflection and recovery of the Cell Index shown in response to control 300 nM 2AT (FIG. 1A) or 30 nM (FIG. 1B) represents a typical EC₅₀ response. High concentrations of Compound I-1 reduced the response, consistent with a partial antagonism.

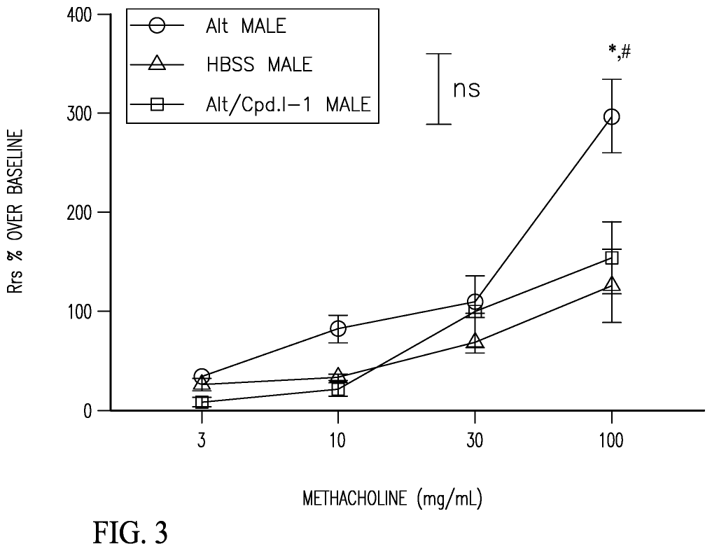

It is known that two primary signaling outcomes of PAR₂ activation are the initiation of Gq/Ca²⁺ and β-arrestin/MAPK signaling. To determine whether the observed in vitro physiologic antagonism represented partial antagonism or intracellular signaling dependent antagonism, digital imaging microscopy was used to assay intracellular Ca²⁺ changes ([Ca²⁺ ]ᵢ; see FIG. 3) as a measure of Gq/Ca²⁺ signaling and an In Cell Western approach for measuring phosphorylation of extracellular regulated kinase ½ (ICW of pERK ½; see FIG. 1D) as an assay for β-arrestin/MAPK signaling. Antagonism at the Gq/Ca²⁺ signaling pathway was not observed, but significant antagonism of the β-arrestin/MAPK-signaling pathway was observed. It is concluded that Compound I-1 is a β-arrestin/MAPK biased PAR₂ antagonist.

### Example 3: In Vivo Pharmacokinetics of Compound I-1

Compound I-1 was evaluated for its plasma stability and oral bioavailability. Mice were fasted overnight before dosing, with water provided ad libitum throughout the study. Three animals are dosed via gavage needle for oral administration at 10 mg/kg (10 mL/kg), via tail vein injection for intravenous administration at mg/kg (2 mL/kg), and via injection in the lower right quadrant of the abdomen for intraperitoneal injections at 10 mg/kg (10 mL/kg). Blood samples (25-30 μL per sample) were taken via saphenous or submandibular vein at 5, 15, 30 min, and 1, 2, 4, 6, 8, 24 h post dose and drug concentration determined by LC-MS/MS. Plasma concentrations were then graphed vs time and pharmacokinetic parameters (terminal half life, t_{1/2}; initial plasma concentration, Cₒ; area under the curve (AUC); volume of distribution at steady-state. Vₛₛ; total plasma clearance CLₚ; mean residence time, MRT; maximum plasma concentration Cₘₐₓ; time to reach Cₘₐₓ, tₘₐₓ; and bioavailability, F) were determined using non-compartmental analysis.

Table 1 below summarizes the pharmacokinetic data for Compound I-1.

TABLE 1

| Cpd 1-1 | Co/Cₘₐₓ | Tₘₐₓ | Cₗₐₛₜ (24 h) | AUC | T_{1/2} | | F | Vss | CLp |
|---|---|---|---|---|---|---|---|---|---|
| (2 mg/kG) | 1.33 | n/a | 1.15 | 595 | 7.9 | h | n/a | 8.29 | 56.6 |
| IV | (μg/mL) | | (ng/mL) | (h/ng/mL) | | | | (L/kg) | (ml/min/kg) |
| (10 mg/kG) | 632 | 30 min | 11.4 | 2520 | 7.45 | h | 84.7% | — | — |
| Oral | (ng/mL) | | (ng/mL) | (h/ng/mL) | | | | | |
| (10 mg/kG) | 689 | 15 min | 12.9 | 2672 | 7.97 | h | 89.8% | — | — |
| IP | (ng/mL) | | (ng/mL) | (h/ng/mL) | | | | | |

### Example 4: In Vivo Efficacy of Compound I-1 in Preventing Stress-Induced Migraine

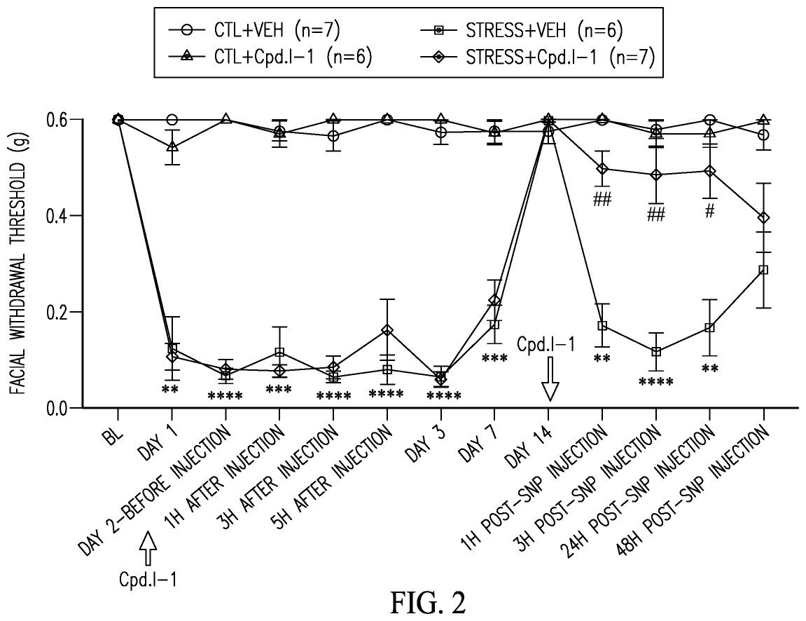

Migraine headaches are characterized by sensitivity of pain-sensing neurons in the dura mater to innocuous stimuli, e.g., the nitric oxide donor, sodium nitroprusside (SNP). Compound I-1 was evaluated in vivo using a mouse migraine model to assess whether it can be used to treat stress-induced migraine-like pain. FIG. 2 summarizes the results for this in vivo study.

Mice were subjected to restraint stress for 3 days (□ and ⋄ data points in FIG. 2) or left unrestrained (∘ and Δ data points in FIG. 2), resulting in a significant facial withdrawal response up to 3 days post-stress. After a 14-day recovery, administration of SNP resulted in a marked facial withdrawal response which was eliminated in the mice that were administered Compound I-1. This demonstrates that Compound I-1 prevents the development of dural sensitization that is associated with chronic migraine pain and has promise as a migraine therapeutic.

### Example 5: In Vivo Efficacy of Compound I-1 in Reducing Allergen-Induced Asthma

Alternaria alternata is a fungal allergen that activates PAR2 in the airways to promote inflammation and bronchoconstriction. Compound I-1 was evaluated in vivo to assess whether it can be used to treat airway hyperresponsiveness induced by Alternaria alternata. FIG. 3 summarizes the results for this in vivo study.

Alternaria alternata or saline (A data points in FIG. 3) were administered intranasally in a mouse asthma model three times over an 8 day period, in the presence (Q data points in FIG. 3) or absence (o data points in FIG. 3) of Compound I-1. Total airway resistance in response to increasing amounts of methacholine was monitored using the FLEXIVENT® (Scireq, Quebec, Canada) system. The results in FIG. 3 suggest that Compound I-1 has promise as a therapeutic for reducing allergen-induced asthma attacks.

Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following Structure (I): 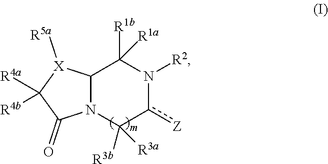
or a stereoisomer or salt thereof, wherein:
R^{1a} and R^{1b} are, each independently, hydrogen, C₁-C₆ alkyl, C₁-C₆ heteroalkyl, C₃-C₈ cycloalkyl, aryl, or arylalkyl;
R² is hydrogen, C₁-C₆ alkyl, C₁-C₆ heteroalkyl, C₃-C₈ heterocyclylalkyl, C₃-C₈ heterocyclylalkylC₁₋₆alkyl, heteroaryl, heteroarylalkyl, or arylalkyl;
Z is either i) oxygen when [symbol] is a double bond or ii) hydrogen, C₁-C₆ alkyl, C₁-C₆ heteroalkyl, C₃-C₈ cycloalkyl, or arylalkyl when [symbol] is a single bond;
R^{3a} and R^{3b} are, each independently, hydrogen, C₁-C₆ alkyl, C₁-C₆ heteroalkyl, C₃-C₈ cycloalkyl, or arylalkyl;
X is N or C(R^{5b});
either
i) R^{4a} and R^{4b} are, each independently, hydrogen, C₁-C₆ alkyl, C₁-C₆ heteroalkyl, C₃-C₈ cycloalkyl, C₃-C₈ heterocyclylalkyl, arylalkyl, or heteroarylalkyl; and
R^{5a} and R^{5b} are, each independently, hydrogen, C₁-C₆ alkyl, C₁-C₆ hydroxyalkyl, C₃-C₈ cycloalkyl, C₃-C₈ heterocyclylalkyl, arylalkyl, heteroarylalkyl, —C(═O)R⁶, —C(═O)YR⁶, —OC(═O)R⁶, —OC(═O)YR⁶, —NHC(═O)R⁶, or —NHC(═O)YR⁶, provided that when R^{5a} and R^{5b} are, each independently, —NHC(═O)R⁶, or —NHC(═O)YR⁶, R⁶ is not an arylalkyl; or
ii) R^{4a} forms a direct bond with X and R^{4b} and R^{5b} together form a 5-9 member fused ring;
R⁶ is C₃-C₈ cycloalkyl, C₃-C₈ heterocyclylalkyl, arylalkyl, heteroarylalkyl, or a 5-9 member fused ring;
Y is O, S, or NR⁷;
R⁷ is hydrogen, hydroxyl, or C₁-C₆ alkyl;
m is 1, or 2; and
wherein each of R^{1a}, R^{1b}, R², R^{3a}, R^{3b}, R^{4a}, R^{4b}, R^{5a}, R^{5b}, R⁶, and R⁷ is optionally substituted with one or more substituent.

2-11. (canceled)

12. The compound of claim 1, wherein the compound having one of the following Structures (Ia)-(Id): 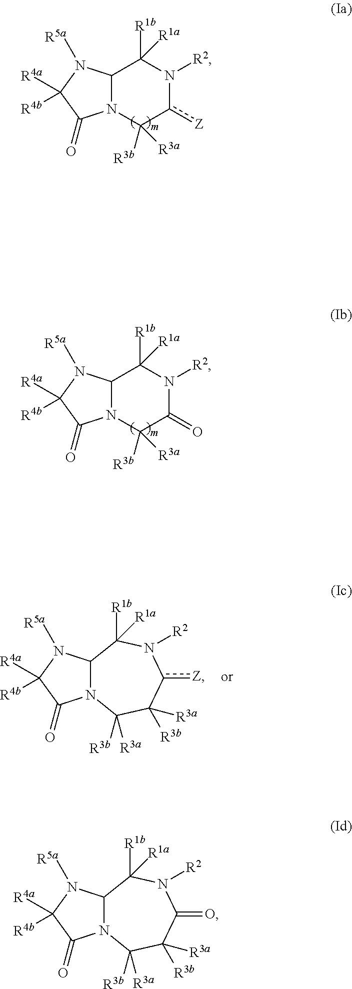
or a stereoisomer or salt thereof.

13. The compound of claim 1, wherein X is C(R^{5b}).

14. (canceled)

15. The compound of claim 1, wherein the compound having one of the following Structures (Ie)-(Ih): 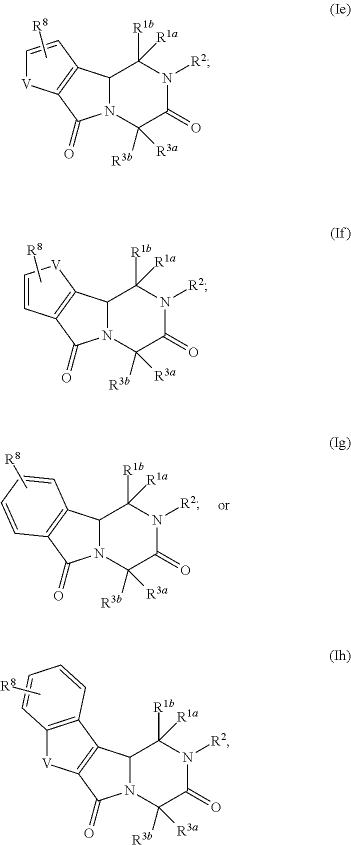
or a stereoisomer or salt thereof, wherein
R⁸ is hydrogen, halo, hydroxyl, alkoxy, amino, cyano, C₁-C₆ alkyl, or C₁-C₆ heteroalkyl; and
V is a carbon, nitrogen, or oxygen atom.

16. The compound of claim 1, wherein at least one of R^{1a} or R^{1b} is hydrogen.

17. The compound of claim 1, wherein each of R^{1a} and R^{1b} are hydrogen.

18. The compound of claim 1, wherein R^{1a} and R^{1b} have one of the following structures: 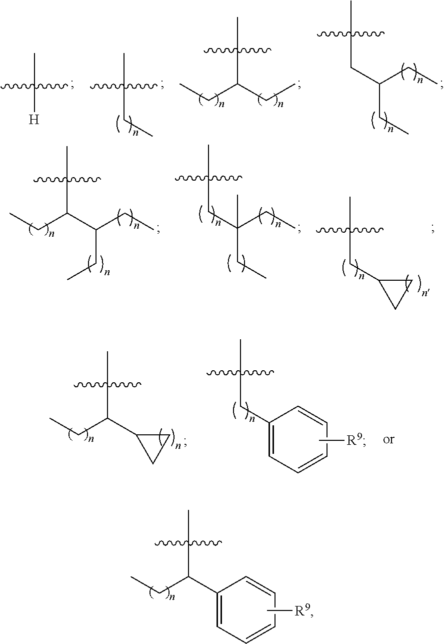
wherein
n is, each independently, 0-6 and n′ is 1-5; and
R⁹ is hydrogen, halo, hydroxyl, alkoxy, amino, cyano, C₁-C₆ alkyl, or C₁-C₆ heteroalkyl.

19. The compound of claim 1, wherein R² has one of the following structures: 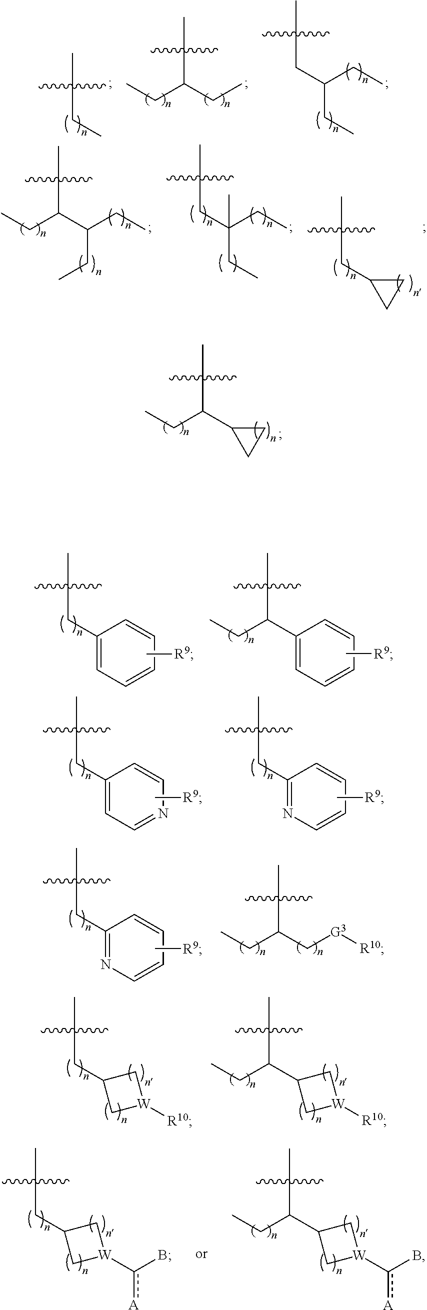
wherein
n is, each independently, 0-6 and n′ is 1-5;
G² is CH₂, NH, O or S;
W is CH or N;
A is O, NH, or NHR⁹, wherein i) [symbol] is a double bond when A is O or ii) [symbol] is a single bond when A is NH or NHR⁹;
B is OR⁹, NH₂, or NHR⁹;
R⁹ is hydrogen, halo, hydroxyl, alkoxy, amino, cyano, C₁-C₆ alkyl, or C₁-C₆ heteroalkyl; and
R¹⁰ is either i) absent or ii) hydrogen, alkyl, aryl, pyridyl, acylalkyl, or acylaryl.

20. The compound of claim 1, wherein R² is

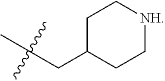 [SMILES: *CC1CCNCC1]

21. The compound of claim 1, wherein R^{3a} or R^{3b} has one of the following structures: 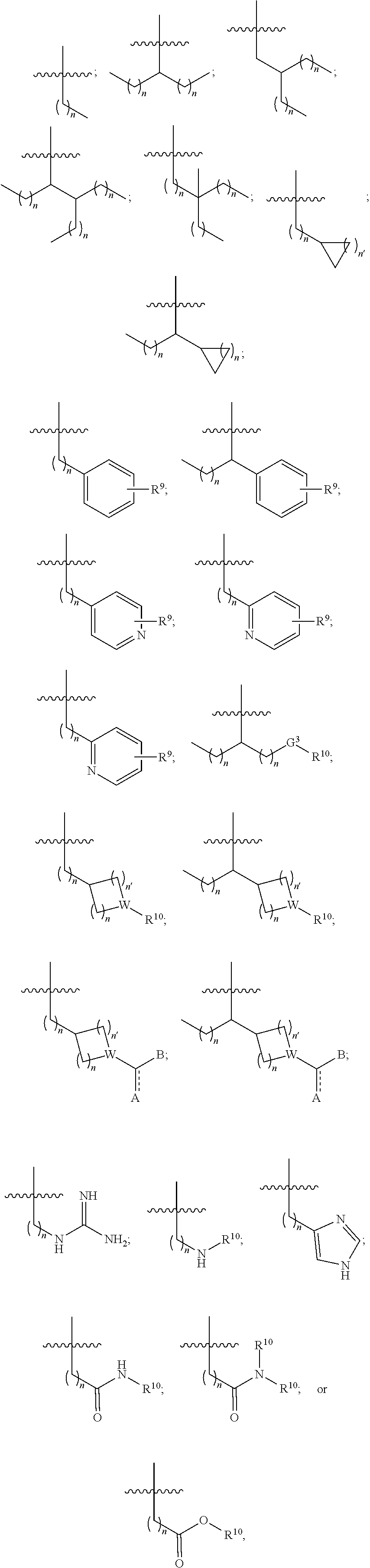
wherein
n is, each independently, 0-6 and n′ is 1-5;
G³ is, each independently, C(R⁷)₂, O, S, or NR⁷;
W is, each independently, CH or N;
A is O, NH, or NHR⁹, wherein i) [symbol] is a double bond when A is O or ii) [symbol] is a single bond when A is NH or NHR⁹;
B is OR⁹, NH₂, or NHR⁹;
R⁹ is hydrogen, halo, hydroxyl, alkoxy, amino, cyano, C₁-C₆ alkyl, or C₁-C₆ heteroalkyl; and
R¹⁰ is either i) absent or ii) hydrogen, alkyl, aryl, pyridyl, acylalkyl, or acylaryl.

22. The compound of claim 1, wherein one of R^{3a} or R^{3b} is hydrogen and the other one of R^{3a} or R^{3b} is

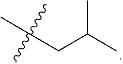 [SMILES: *CC(C)C]

23. The compound of claim 1, wherein one of R^{3a} or R^{3b} is hydrogen and the other one of R^{3a} or R^{3b} is

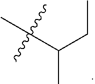 [SMILES: *C(C)CC]

24. The compound of claim 1, wherein R^{4a} or R^{4b} has one of the following structures: 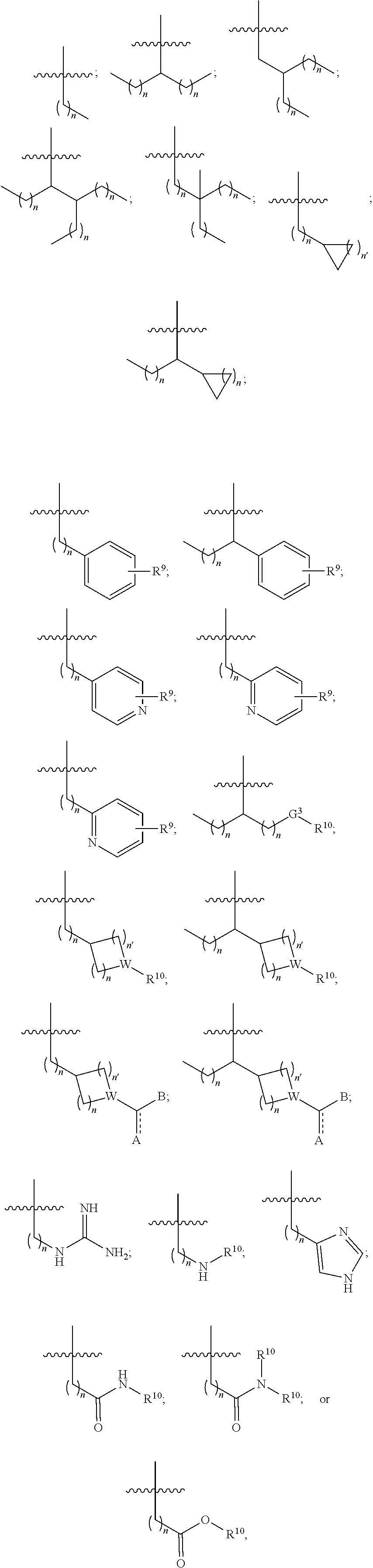
wherein
n is, each independently, 0-6 and n′ is 1-5;
G⁴ is, each independently, C(R⁷)2, O, S, or NR⁷;
W is, each independently, CH or N;
A is O, NH, or NHR⁹, wherein i) [symbol] is a double bond when A is O or ii) [symbol] is a single bond when A is NH or NHR⁹;
B is OR⁹, NH₂, or NHR⁹;
R⁹ is hydrogen, halo, hydroxyl, alkoxy, amino, cyano, C₁-C₆ alkyl, or C₁-C₆ heteroalkyl; and
R¹⁰ is either i) absent or ii) hydrogen, alkyl, aryl, pyridyl, acylalkyl, or acylaryl.

25. The compound of claim 1, wherein one of R^{4a} or R^{4b} is hydrogen and the other one of R^{4a} or R^{4b} is

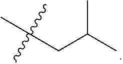 [SMILES: *CC(C)C]

26. The compound of claim 1, wherein R^{5a} has one of the following structures: 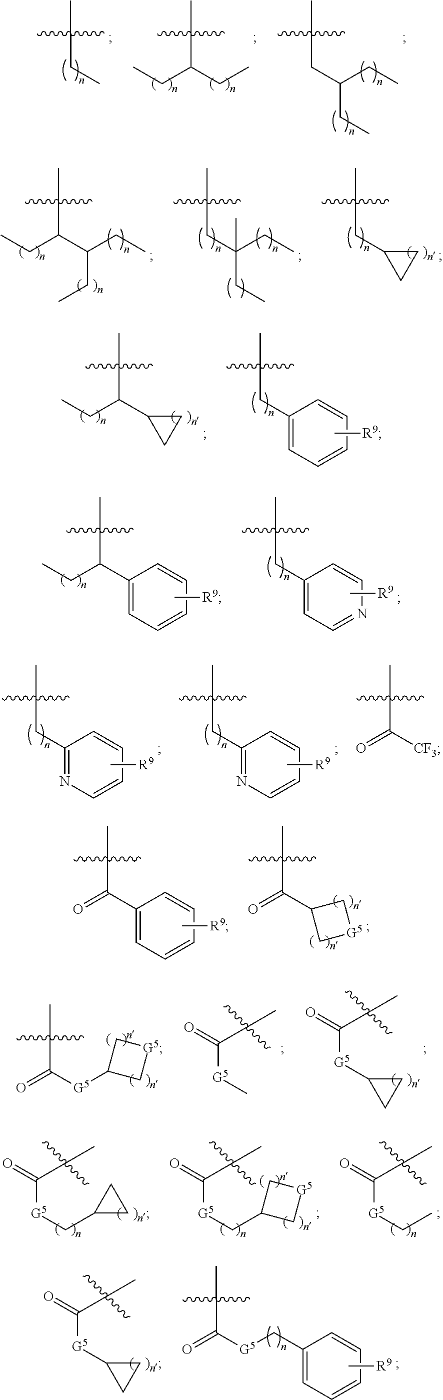 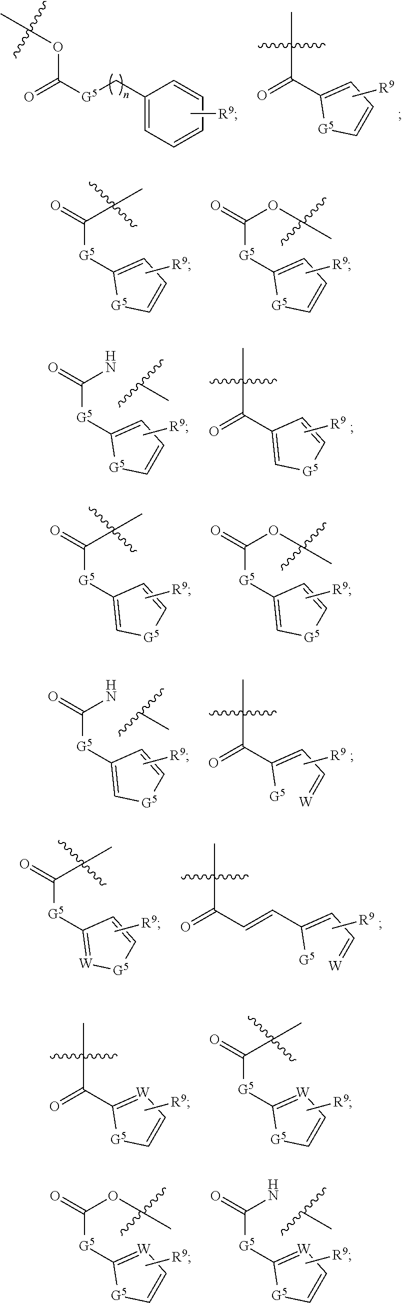 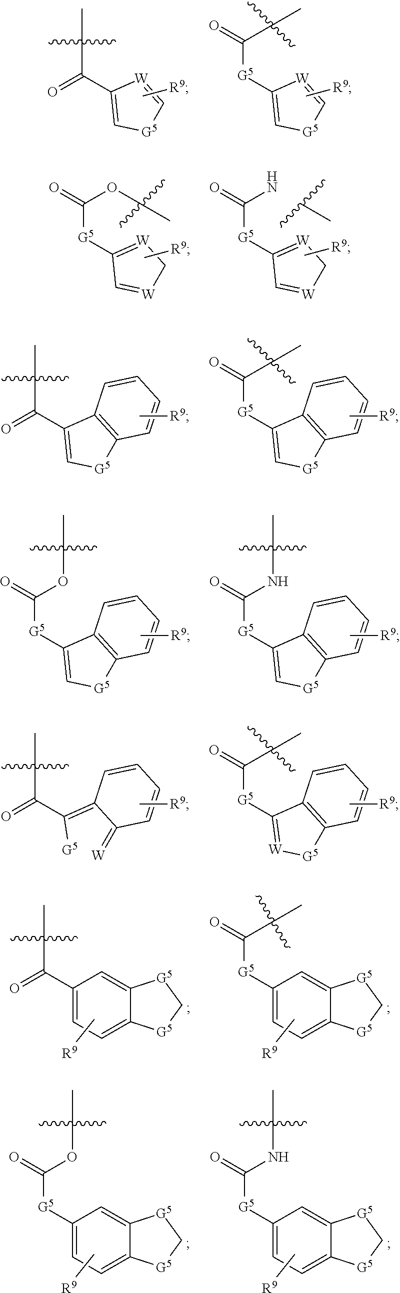 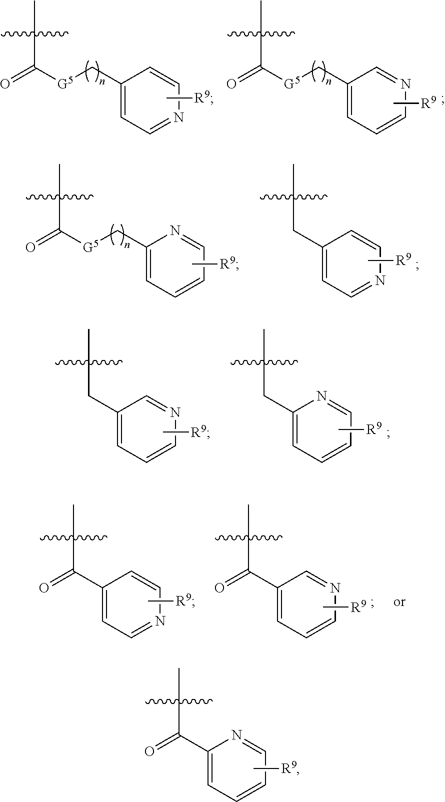
wherein
n is 0-6 and n′ is 1-5;
G⁵ is, each independently, C(R⁷)₂, O, S, or NR⁷; and
W is, each independently, CH or N.

27. The compound of claim 1, wherein R^{5a} is 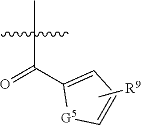
and R^{5b} is hydrogen.

28. The compound of claim 27, wherein W is O and R⁹ is hydrogen.

29. The compound of claim 1, wherein R^{5a} has one of the following structures:

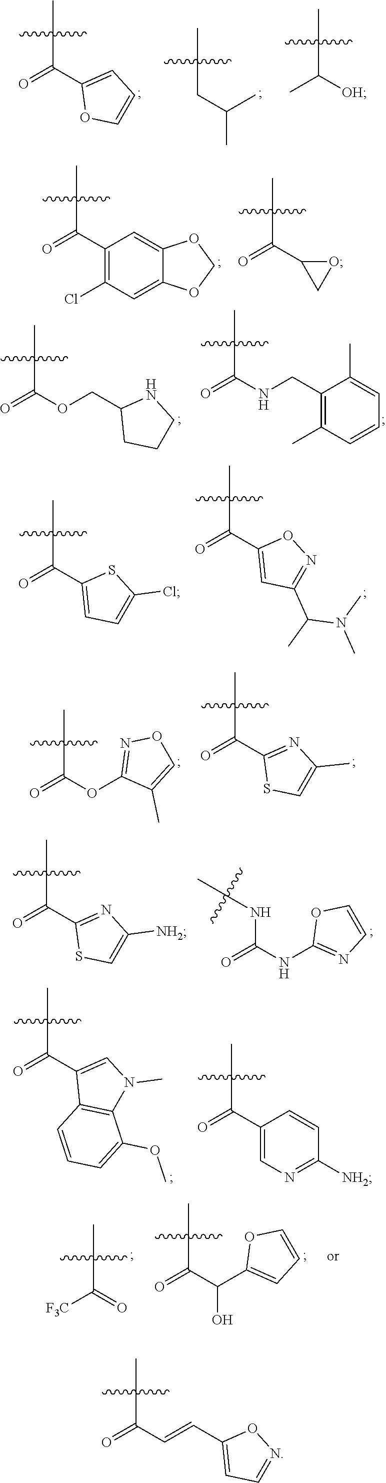

30. The compound of claim 1, wherein the compound has one of the following structures: 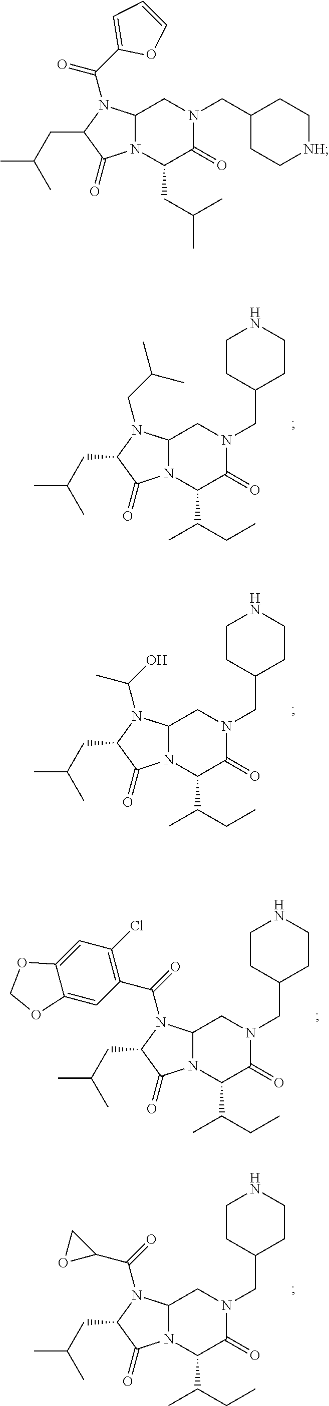 [SMILES: CC(C)CC1C(=O)N2C(CN(CC3CCNCC3)C(=O)[C@@H]2CC(C)C)N1C(=O)c1ccco1 ; CCC(C)[C@H]1C(=O)N(CC2CCNCC2)CC2N(CC(C)C)[C@@H](CC(C)C)C(=O)N21 ; CCC(C)[C@H]1C(=O)N(CC2CCNCC2)CC2N1C(=O)[C@H](CC(C)C)N2C(C)O ; CCC(C)[C@H]1C(=O)N(CC2CCNCC2)CC2N1C(=O)[C@H](CC(C)C)N2C(=O)c1cc2c(cc1Cl)OCO2 ; CCC(C)[C@H]1C(=O)N(CC2CCNCC2)CC2N1C(=O)[C@H](CC(C)C)N2C(=O)C1CO1] 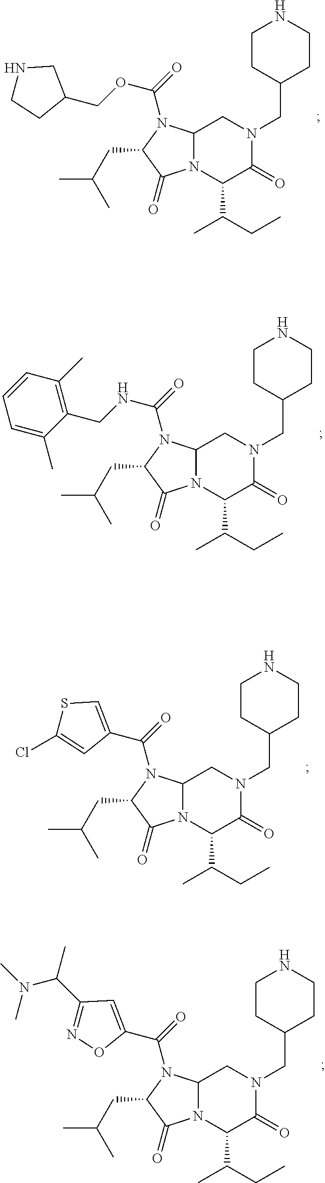 [SMILES: CCC(C)[C@H]1C(=O)N(CC2CCNCC2)CC2N1C(=O)[C@H](CC(C)C)N2C(=O)OCC1CCNC1 ; CCC(C)[C@H]1C(=O)N(CC2CCNCC2)CC2N1C(=O)[C@H](CC(C)C)N2C(=O)NCc1c(C)cccc1C ; CCC(C)[C@H]1C(=O)N(CC2CCNCC2)CC2N1C(=O)[C@H](CC(C)C)N2C(=O)c1csc(Cl)c1 ; CCC(C)[C@H]1C(=O)N(CC2CCNCC2)CC2N1C(=O)[C@H](CC(C)C)N2C(=O)c1cc(C(C)N(C)C)no1] 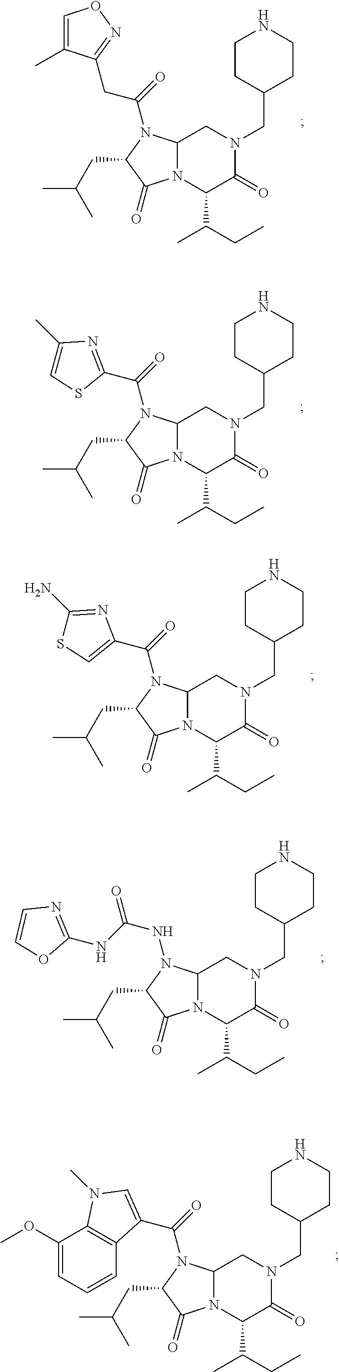 [SMILES: CCC(C)[C@H]1C(=O)N(CC2CCNCC2)CC2N1C(=O)[C@H](CC(C)C)N2C(=O)Cc1nocc1C ; CCC(C)[C@H]1C(=O)N(CC2CCNCC2)CC2N1C(=O)[C@H](CC(C)C)N2C(=O)c1nc(C)cs1 ; CCC(C)[C@H]1C(=O)N(CC2CCNCC2)CC2N1C(=O)[C@H](CC(C)C)N2C(=O)c1csc(N)n1 ; CCC(C)[C@H]1C(=O)N(CC2CCNCC2)CC2N(NC(=O)Nc3ncco3)[C@@H](CC(C)C)C(=O)N21 ; CCC(C)[C@H]1C(=O)N(CC2CCNCC2)CC2N1C(=O)[C@H](CC(C)C)N2C(=O)c1cn(C)c2c(OC)cccc12] 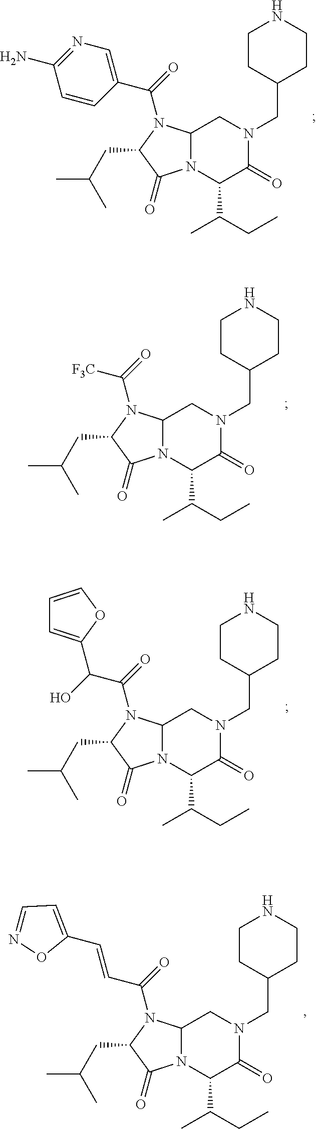 [SMILES: CCC(C)[C@H]1C(=O)N(CC2CCNCC2)CC2N1C(=O)[C@H](CC(C)C)N2C(=O)c1ccc(N)nc1 ; CCC(C)[C@H]1C(=O)N(CC2CCNCC2)CC2N1C(=O)[C@H](CC(C)C)N2C(=O)C(F)(F)F ; CCC(C)[C@H]1C(=O)N(CC2CCNCC2)CC2N1C(=O)[C@H](CC(C)C)N2C(=O)C(O)c1ccco1 ; CCC(C)[C@H]1C(=O)N(CC2CCNCC2)CC2N1C(=O)[C@H](CC(C)C)N2C(=O)/C=C/c1ccno1]
or a stereoisomer or salt thereof.

31. The compound of claim 1, wherein the compound is a pharmaceutically acceptable salt.

32. A pharmaceutical composition comprising a compound or salt of claim 1 and a pharmaceutically acceptable carrier.
